(19) United States
(12) Patent Application Publication
(10) Pub. No.: US 2021/0016087 A1
(43) Pub. Date: Jan. 21, 2021

(54) OCCLUSION THERAPY AND PELVIC STIMULATION SYSTEM

(71) Applicant: CYMEDICA ORTHOPEDICS, INC., Scottsdale, AZ (US)

(72) Inventors: Kereshmeh Shahriari, Cave Creek, AZ (US); Struan H. Coleman, Locust Valley, NY (US); Answorth Allen, New York, NY (US)

(21) Appl. No.: 16/932,554

(22) Filed: Jul. 17, 2020

Related U.S. Application Data
(60) Provisional application No. 62/875,240, filed on Jul. 17, 2019.

(51) Int. Cl. A61N 1/36; A61N 1/04

(52) U.S. Cl. CPC A61N 1/36007; A61N 1/36031; A61N 1/0484

(57) ABSTRACT

The system is directed to improving muscle strength using electrostimulation therapy and/or occlusion according to some embodiments. In some embodiments, the system is configured to use electrodes placed outside the body and/or on the surface of the skin to activate pelvic floor muscles to control urine flow from the bladder. In some embodiments, the system includes one or more fluid bladders configured to reduce blood flow to a targeted area while applying electrostimulation using one or more electrodes. In some embodiments, the occlusion system results in higher muscle growth and/or retraining results than conventional methods.

### CROSS REFERENCE

This application claims priority to U.S. Provisional Patent Application No. 62/875,240, filed Jul. 17, 2019, the entire contents of which are hereby incorporated.

### BACKGROUND

Stress urinary incontinence, overactive bladder, or spastic bladder refers to involuntary loss of urine due to inadequate urethral pressure. The patients experience urine loss during coughing, sneezing, or physical exertion.

Patients with the stress urinary incontinence disease are not able to effectively contract the pelvic floor muscles during increased bladder pressure due to weak pelvic floor muscles. In some patients, the lower pelvic muscles can become damaged or weakened through childbirth, lack of use, aging, or as a result of surgical procedures. In some patients, one symptom related to a weakening of these muscles can be urinary incontinence. In some patients, pelvic disorders can include chronic pelvic pain and vulvodynia (i.e., pelvic muscle dysfunction) that can be experienced by young adult women. In some patients, these disorders can be caused by involuntary contractions (i.e., spasms) of the levator aniand perineal muscles.

Conventional approaches to the treatment of stress urinary incontinence include one or more of: behavioral modifications, physical therapy, drug-based therapies, surgery, and electrical stimulation of sacral nerve through implantable electrodes or stimulators which include the use of internal probes (i.e., transvaginal or rectal approach). Implantable electrodes have also been used to stimulate the pelvic floor muscles directly. It is well known that incidence of unintentional episodes of bladder emptying can be reduced by using these techniques. However, the drawback for these traditional neuromuscular electrical stimulation therapeutic methods can be one or more of: risk associated with surgery, discomfort and pain associated with internal probes, lack of feedback for the actual amount of electrical stimulation applied, and/or a poor patient compliance.

Gluteus weakness or atrophy is a common disease resulting from the hip osteoarthritis, or a hip surgical procedure such as hip arthroscopy or hip replacement, or gluteal nerve damage, or lack of activity. The patients experience imbalance, difficulty climbing the stairs, hip or knee pain, lower back pain, or abnormal gait.

Lower back pain is a very common and disabling condition with significant financial and social costs. Lifetime prevalence is extremely high, with 60-80% of people experiencing low back pain and related conditions at least some time in their lives. It all too frequently persists to cause chronic problems for sufferers.

The deep local muscles of the lumbopelvic region can contribute to the stability of the spinal column and can be implicated in the development of low back pain. In some embodiments, atrophy changes can affect the lumbar multifidus and the motor control disturbances. Atrophy changes can affect the internal oblique and transversus abdominis. The atrophy changes can result in segmental instability of the lumbar spine and can cause or perpetuate low back pain. A decrease in contractile tissue through fatty infiltration or atrophy can reduce the force generation capabilities of the lumbar multifidus which can affect the muscle's role as a spinal stabilizer.

Conventional methods to treat these disorders include exercise and physical therapy. Corrective exercise programs can address atrophy changes in the lumbar multifidus, and abdominal muscle motor control disturbances is an important component in the management of low back pain. Research has demonstrated specific stabilization exercises targeting the local muscles can result in the improvements of pain and disability. Results include a reduced incidence of recurrence in patients with acute and chronic low back pain. However, this type of rehabilitation can be very labor intensive, require extensive instruction, and can require input from a therapist. In addition, evidence suggest that patients frequently have difficulty initiating contractions of the local muscles on their own, making effective rehabilitation challenging.

Transcutaneous electrical nerve stimulation is a commonly employed electrical modality in the treatment of low back pain. Transcutaneous electrical nerve stimulation can be effective in facilitating short term improvements in pain related disability and can be a passive intervention that does not target the underlying muscle dysfunction. Cortisone injections or numbing medication into the epidural space is also commonly prescribed. A cortisone injection can help decrease inflammation around the nerve roots. However, the pain relief generally lasts less than a few months.

Conventional studies have found that blood flow restricted training, the brief and partial restriction of venous outflow of an extremity during low load resistance exercises, an effective method of improving strength in healthy, active individuals. Studies have shown that occlusion techniques during exercise allows for a lower weight training regimen to build an equal amount of muscle that would be obtained at higher weights. An unexplored potential of this adjunctive modality lies in treating patients with severe musculoskeletal trauma, persistent chronic quadriceps and hamstring weakness despite traditional therapy, and low improvement during early postoperative strengthening. However, conventional techniques that include placing a tourniquet on a limb and occluding a percentage of arterial inflow and venous outflow can induce a metabolic stress.

Therefore, it would be desirable to have a system that is able to stimulate the sacral nerve and/or pelvic floor muscle externally while monitoring effectiveness to eliminate the need for internally placed probes. It would also be desirable to have an occlusion device capable of restricting blood flow while simultaneously applying and/or measuring electrical stimulation.

### SUMMARY

In some embodiments, the system described herein comprises one or more wraps, garments, bands, and/or or belts that each include one or more electrodes, sensors, and/or occlusion systems.

In some embodiments, the system includes at least one sensor comprising a plurality of electrodes including at least one active electrode and at least one receiving electrode, the at least one sensor configured and arranged to be in physical contact with skin of a patient forming an electrical circuit with control electronics of at least one controller, the electrical circuit configured and arranged to measure an electrical parameter using the at least one active electrode and at least one receiving electrode, and to form a closed loop electrical muscle stimulation system. In some embodiments, a stimulation current or voltage applied by the sensor onto the skin between the at least one active electrode and at least one receiving electrode is based on at least one program and at least one electrical parameter measured through the at least one active electrode and at least one receiving electrode. In some embodiments, the at least one controller configured to execute one or more of the following instructions:
(a) apply a sense electrical pulse to the tissue using the at least one sensor,
(b) measure the at least one electrical parameter from the tissue,
(c) using at least one of the active electrodes, adjustably apply a stimulation pulse to the tissue based at least in part on the measured electrical parameter, the stimulation being adjustably controlled by the at least one controller to maintain a constant power output to the tissue based at least in part on the at least one electrical parameter, and
(d) repeat steps (a)-(c).

In some embodiments, the pubococcygeal muscle or pelvic floor muscle can be responsible for holding the pelvic organs within the pelvic cavity. In some embodiments, the pelvic floor muscle can consist of a deep muscle layer and a superficial muscle layer. In some embodiments, the deep muscle layer and superficial muscle layer can work together to keep the pelvic organs healthy and in good working order. In some embodiments, the muscle can be suspended like a hammock at the base of the pelvis. In some embodiments, the muscle can wrap around the vagina and rectum in an over-under figure eight pattern.

In some embodiments, the system is configured to stimulate the pelvic floor muscle to create a contraction of these muscles to re-educate and/or treat weakness or disuse atrophy associated with stress urinary incontinence syndrome. In some embodiments, the system is configured to improve partially de-nervated urethral and pelvic floor musculature by enhancing the process of re-innervation via pelvic floor muscle stimulation. In some embodiments, the system includes one or more of neuromuscular electrical stimulation delivery, real-time EMG detection, a patient mobile app, neuromuscular electrical stimulation management, low back pain related clinical surveys, neuromuscular electrical stimulation compliance, machine learning methods for personalized neuromuscular electrical stimulation dose, and a provider portal. In some embodiments, the neuromuscular electrical stimulation is configured to improve gluteus muscle strength.

In some embodiments, the neuromuscular electrical stimulation therapy is configured to be applied non-invasively from outside the pelvis region using cutaneous surface electrodes. In some embodiments, electrodes placed externally over the pelvis region are configured to stimulate the pudendal nerve and at least partially cause urethral closure by activating the pelvic-floor musculature. In some embodiments, the cutaneous surface electrodes are configured to stimulate the motor neurons of the pudendal nerve and pelvic floor muscles. In some embodiments, the system is configured to apply neuromuscular electrical stimulation therapy non-invasively by stimulating muscles of the back and abdominal wall. In some embodiments, the neuromuscular electrical stimulation therapy stimulates the gluteus muscles including one or more of the gluteus maximum and/or gluteus medius. In some embodiments, the system is configured to create contractions of the gluteus muscles to treat, re-educate, and/or rebuild the gluteus muscles over a period of time. In some embodiments, the system is configured to treat atrophy associated with hip osteoarthritis, hip surgical procedures, and/or any other clinical condition associated with gluteus weakness.

In some embodiments, the system comprises electrode flaps and/or hook and loop fasteners configured to secure one or more electrodes and/or sensors assemblies and/or enable replacement of one or more electrodes and/or sensors assembly components. In some embodiments, the system is configured to provide neuromuscular electrical stimulation waveform, closed-loop feedback, and power-controlled output using one or more electrodes and/or sensors.

In some embodiments, the stimulation electrodes and/or sensors assembly (i.e., one or more sensor pairs) is provided in a wrap, garment, shorts, and/or individual patches for each leg. In some embodiments, the wrap, garment, and/or shorts can include two to six pre-positioned electrodes. In some embodiments, the two to six electrodes are configured and arranged to stimulate one or more of the anterior, posterior, and/or lateral regions of the pelvis. In some embodiments, the system includes one or more electrodes that cover a combined region between 60% and 100% of the total amount of user's skin that covers the gluteal muscles. In some embodiments, system comprises one or more of two electrodes positioned on the right gluteal maximus, two electrodes positioned on the left gluteal maximus, one electrode positioned on the right gluteal medius, and/or one electrode positioned on the left gluteal medius. In some embodiments, he sensors are configured to provide real-time stimulation biofeedback to the patient by measuring the intensity of the muscle contractions to measure the effectiveness of the muscle contraction. In some embodiments, the system includes one or more garments configured to be wearable by a user, the garments comprising at least one top opening configured to surround a user's waist, the garment comprising at least two bottom openings located distal from the top openings, the at least two bottom openings each configured to surround a user's leg.

In some embodiments, the stimulation electrodes and sensors assembly are provided in a wrap, garment, shorts, or individual patches for each leg. In some embodiments, at least one sensor is configured and arranged to at least partially cover a user's gluteus muscles. In some embodiments, the stimulation electrodes, EMG electrodes, and/or pressure and/or force gauge sensors are configured to be detachable components and/or are embedded in a conductive textile. In some embodiments, the system is configured to provide real-time muscle contraction measurements and user feedback related to the neuromuscular electrical stimulation usage, EMG feedback, and/or muscle contraction intensities.

In some embodiments, the system comprises one or more of a wrap, garment, band, and/or belt (hereafter collectively referred to as a garment) for treatment of low back pain stimulation. In some embodiments, stimulation electrodes are placed in the belt for lower back pain treatment. In some embodiments, cutaneous surface electrodes (i.e., electrodes) are placed externally over the lumbopelvic region. In some embodiments, the electrodes are configured to stimulate the motor neurons of the posterior primary divisions of the spinal nerves and deep lumbar stabilizing muscles of the spine. In some embodiments, the system includes two to eight electrodes configured to stimulate the lower back and/or abdominal walls. In some embodiments, the electrodes are one or more of embedded conductive textile and/or attachable disposable electrodes.

In some embodiments, the system includes one or more of neuromuscular electrical stimulation waveform, closed-loop feedback, and power-controlled output. In some embodiments, the system is configured to measure the intensity of the muscle contractions and provide real-time stimulation biofeedback to the patient. In some embodiments, one or more of the electrodes, force gauge sensors, electromyography (EMG) electrodes (i.e., EMG sensors) are incorporated into the same electrostimulation garment. In some embodiments, the electrodes, force gauge sensors, electromyography (EMG) electrodes are each detachable components of the system and/or are embedded in garment as conductive textile.

In some embodiments, the stimulation electrodes and sensors are provided in a garment configured to cover at least a portion of the lower back and abdominal area. In some embodiments, the system provides neuromuscular electrical stimulation therapy that is configured to stimulate the transversus abdominis, lumbar multifidus, and the internal and external oblique muscles. In some embodiments, the neuromuscular electrical stimulation therapy is configured to stimulate the deep local muscles of the lumbopelvic region.

In some embodiments, the surface electrodes are placed on the patient's low back (L5-S1 spinous process) and/or abdominal wall. In some embodiments, the electrodes are placed externally over the lower back and/or abdominal region of the body and are configured to stimulate the posterior primary divisions of the spinal nerves. In some embodiments, the system is configured to stimulate the lumbopelvic region muscles. In some embodiments, the system is configured to create a contraction of muscles configured to re-educate and/or treat the weakness or disuse atrophy associated with lower back pain over time.

In some embodiments, wherein the system comprises a blood flow restriction system comprising one or more garments that include one or more fluid pressure bladders, wraps, belts, and/or cuffs. In some embodiments, one or more garments include both an electrode muscle stimulation system and blood flow restriction system. In some embodiments, the electrode muscle stimulation system includes one or more electrodes and/or enabling circuits described herein. In some embodiments, the blood flow restriction system includes one or more fluid bladders and/or fluid delivery components described herein.

In some embodiments, one or more blood flow restriction fluid bladders are positioned on top of the electrical muscle stimulation electrode such that the electrical muscle stimulation electrode is positioned between the user's skin and the one or more blood flow restriction fluid bladders. In some embodiments, the fluid bladder is positioned inside at least a portion of a garment. the blood flow restriction system is configured to apply electrical muscle stimulation therapy. In some embodiments, the blood flow restriction system includes one or more air channels, ducts, valves and/or tubes configured to enable fluid flow and/or prevent fluid flow. In some embodiments, blood flow restriction system includes air channels, ducts and/or tubes configured to enable occlusion to one or more portions of one or more user limbs. In some embodiments the system includes a blood flow restriction board substrate.

In some embodiments, the system is configured to achieve the blood flow restriction through an applied external pressure over the extremities using one or more fluid bladders coupled to one or more garments. In some embodiments, the applied external pressure is configured to maintain arterial inflow while occluding venous outflow distal to the occlusion site. In some embodiments, the blood flow restriction is configured to create a hypoxic environment in the limb. In some embodiments, the hypoxic environment in the limb is a result of a decreased oxygen supply from less arterial blood flow. In some embodiments, the hypoxic environment causes the brain to respond with a natural repair response similar to high intensity exercises. In some embodiments, the natural repair responses include stimulation one or more of growth hormone, insulin growth factor, protein synthesis, myosatellite (stem) cells, and the like. In some embodiments, the system is configured to trigger a user's body to make new tissue without having to repair any damaged tissue. In some embodiments, the system is configured to trigger a user's body to faster muscle strength and/or size gains with lower loads than required to produce the same strength and/or size gains using resistance weight training only.

In some embodiments, the system is configured to combine electrical muscle stimulation therapy and blood flow restriction in one device. In some embodiments, the electrical muscle stimulation therapy is configured to provide strengthening of the muscles using electrical stimulation. In some embodiments, the system is configured to enhance muscle adaptation and growth. In some embodiments, the electrical muscle stimulation therapy is configured to enhance blood flow circulation and growth hormone release. In some embodiments, clinical applications of the electrical muscle stimulation therapy include post-operative treatment for lower extremity surgeries of the knee and hip.

In some embodiments, the blood flow restriction system includes a pump system that includes at least one pump and/or a small pump controller unit. In some embodiments, the pump is controlled at least in part by the electrical muscle stimulation controller. In some embodiments, the pump is a pneumatic pump.

In some embodiments, blood flow rate is displayed by the pump system as an analog and/or digital display reading. In some embodiments, the display for blood flow rate is an integrated part of a mobile app user interface. In some embodiments, the mobile app interface and/or display on the controller is configured to enable pressurizing control of the garment. In some embodiments, the system includes a wireless connection between the pneumatic pressure controller and the mobile app display, the wireless connection comprising one or more of Bluetooth® or WiFi® protocol.

In some embodiments, the system is configured to apply a maximum collusion pressure that results in no injury. In some embodiments, the mobile app is configured to adjust the pressure range.

In some embodiments, the system includes a doppler ultrasound configured to monitor blood flow. In some embodiments, ultrasound system is configured and arranged to measure the speed of the blood flow in the occlusion area from the outer surface of the skin. In some embodiments, the ultrasound system is configured to display the blood flow on a controller display and/or mobile app display. In some embodiments, the doppler ultrasound system monitors blood flow during one or more of while garment is being pressurized, while garment is being depressurized, while garment is applying pressure.

In some embodiments, the mobile app is configured to enable a user to pressurize the garment and/or cuff when instructed by the app and/or before the electrical muscle stimulation. In some embodiments, the system includes a button on the pressure control unit and/or app configured to control pressure to the garment. In some embodiments, the controller unit and/or mobile app is configured to display the pressure and/or stop the pump at the desired pressure level set by the patient. In some embodiments, wherein the garment is configured to stay inflated for the duration of electrical muscle stimulation therapy. In some embodiments, the garment and/or cuff is configured to be deflated anytime during the electrical muscle stimulation cycle by the patient. In some embodiments, one or more portions of the garment system is configured to apply blood flow restriction and neuromuscular electrical stimulation therapies together and/or separately.

In some embodiments, the system further includes patient facing mobile app as an interface between the neuromuscular electrical stimulation controller and the patient. In some embodiments, the mobile app is configured to provide a tool for the patient to apply and control delivery of neuromuscular electrical stimulation and/or occlusion. In some embodiments, the mobile app is configured to display real-time muscle contractions or EMGs measured by the EMG and/or force gauge sensors. In some embodiments, the mobile app is configured to collect, display, and report a patient's health data. In some embodiments, the mobile app is configured to execute and/or display a patient bladder diary. In some embodiments, the patient bladder diary is configured to record urinary leakage episodes and/or correlate the recorded urinary leakage with daily neuromuscular electrical stimulation usage. In some embodiments, the mobile app is configured to track the use of medications related to stress urinary incontinence conditions. In some embodiments, the mobile app is configured to track the use of medications related to joint and/or pain conditions. In some embodiments, the mobile app is configured to collect multiple clinical surveys, where at least one survey includes a questionnaire comprising questions for one or more of urinary incontinence diagnoses, quality of life, HOOS/HOOS JR. survey, pain survey, and the like.

In some embodiments, the mobile app is configured to enable the patient to apply and control delivery of neuromuscular electrical stimulation. In some embodiments, the mobile app is configured to display real-time muscle contractions or EMGs measured by the EMG or force and/or pressure gauge sensors. In some embodiments, the mobile app is configured to track a patient's daily back pain and correlate it with daily neuromuscular electrical stimulation usage. In some embodiments, the mobile app is configured to adjust the neuromuscular electrical stimulation dosage by one or more of automatically and/or by a user.

In some embodiments, the system a database and a provider facing portal to collect, store, and display the patient's neuromuscular electrical stimulation usage and reported patient outcomes. In some embodiments, the system is configured to enable providers to analyze and review the data and intervene in the patient's care by changing one or more parameter settings remotely. In some embodiments, the system is configured to provide real-time outcome measures and user feedback related to the neuromuscular electrical stimulation usage, EMG feedback, and muscle contraction intensities. In some embodiments, the system is configured to integrate collected data with a patient's smart phone/tablet health kit data such as Apple health data.

In some embodiments, the system comprises machine learning and/or artificial intelligence (AI) configured to provide an optimized and personalized recommended neuromuscular electrical stimulation dose based on one or more of the patient's demographic, neuromuscular electrical stimulation usage, collected clinical conditions through the device sensors, and/or patient reported outcomes. In some embodiments, the machine learning and/or AI is configured to receive real-time outcome measures and user feedback related to the neuromuscular electrical stimulation usage, EMG feedback, and muscle contraction intensities while the system is providing electrical stimulation to the user.

In some embodiments, the system further comprising neuromuscular electrical stimulation generators or controllers. In some embodiments, one or more neuromuscular electrical stimulation generators and/or controllers are placed on the front and/or sides of one or more garments. In some embodiments, the neuromuscular electrical stimulation generators and/or controllers are secured by one or more processes comprising bonding, die cutting, and/or sewing. In some embodiments, the system is configured to provide electrical connectivity among the electrodes, controllers, and/or sensors via one or more of printed conductive textile traces, wires, or wireless communication. In some embodiments, wireless communication includes one or more of Bluetooth®, WiFi®, or the like.

In some embodiments, the system includes one or more accelerometers, gyros, GPS, inertial measurement unit, inertial motion capture sensors that are used in conjunction with the blood flow restriction system and/or neuromuscular electrical stimulation system described herein. In some embodiments, the one or more accelerometers, gyros, GPS, inertial measurement unit, inertial motion sensors (collectively referred to as capture sensors) are positioned in the garment. In some embodiments, the capture sensors are configured to provide measurements of the hip joint kinematics and/or positioning including one or more of: hip ROM, hip gait, hip rotation angle, hip adduct moment, hip contact force, steps, gait, and/or speed. In some embodiments, capture sensors are configured to provide include body temperature, heart rate, muscles EMG, and muscle strength measurements to one or more components of the system.

### DESCRIPTION OF THE DRAWINGS

FIG. 1A is a fragmentary perspective view of a knee brace mounted onto the knee of a patient according to some embodiments.

FIG. 1B is a perspective view of a knee joint according to some embodiments.

FIG. 2 is a fragmentary perspective view of a knee brace mounted onto the knee of a patient according to some embodiments.

FIG. 3 is a block diagram of the knee brace of FIG. 1 in communication with a computer according to some embodiments.

FIG. 4 is a flow diagram of an example of steps performed according to some embodiments.

FIG. 5 is a perspective view of a knee brace according to some embodiments.

FIG. 6 is a perspective view of control electronics of a knee brace according to some embodiments.

FIG. 7 is a perspective view of sensors of the knee brace according to some embodiments.

FIG. 8 is a perspective view of a soft garment connected to a control unit according to some embodiments.

FIG. 9 is a perspective view of a soft garment connected to a control unit in according to some embodiments.

FIG. 10A is a signal diagram illustrating the signals transmitted into the human tissues by the electrodes/sensors according to some embodiments.

FIG. 10B is a flowchart showing steps performed by the control electronics in according to some embodiments.

FIG. 11 is a signal diagram illustrating the propagation delay between the stimulation pulse and the receive pulse transmitted and received by the electrodes/sensors according to some embodiments.

FIG. 12 is a signal diagram illustrating the power supply signal produced by the power supply according to some embodiments.

FIG. 13 is a block diagram of a circuit configured to measure the dynamic properties of the electrodes in a channel according to some embodiments.

FIG. 14 is an analog sense circuit to measure the source voltage and source current according to some embodiments.

FIG. 15 is a circuit diagram of a circuit to generate a stimulation pulse according to some embodiments.

FIG. 16 is a waveform diagram of an input waveform, a desired output waveform, and a target voltage according to some embodiments.

FIG. 17 illustrates an occlusion portion of the system with one or more fluid bladders, sensors, and/or electrodes coupled to a knee brace according to some embodiments.

FIG. 18 illustrates a pelvic stimulation portion of the system according to some embodiments.

FIG. 19 are diagrams showing pelvic area anatomical structures according to some embodiments.

FIG. 20 are diagrams showing pelvic area nerves according to some embodiments.

FIG. 21 illustrates additional anatomical structures according to some embodiments.

FIGS. 22-24 show the occlusion system of FIG. 17 as a wrap according to some embodiments.

FIG. 25 shows sensor pairs 2501, 2502; 2503, 2504 configured and arranged on a user's lower back above the waistline for lower back pain treatment with NMES according to some embodiments.

### DETAILED DESCRIPTION

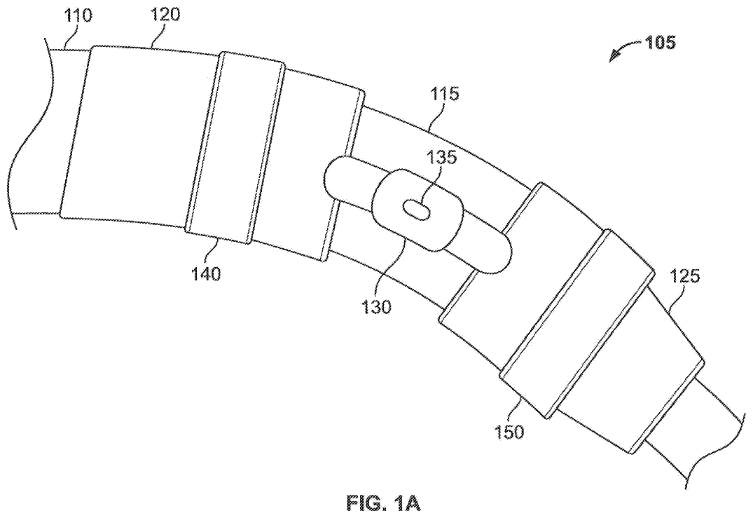

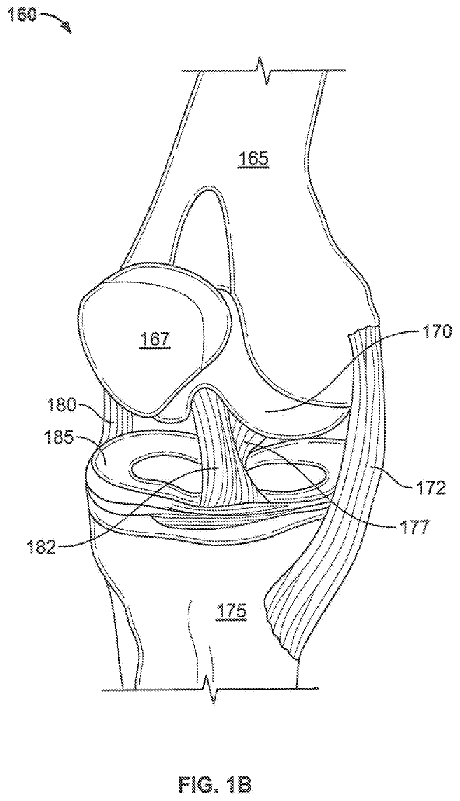

FIG. 1A is a fragmentary perspective view of a knee brace 105 configured to be mounted onto the leg 110 of a person/patient according to some embodiments. In some embodiments, the brace 105 is intended to control movement of the thigh to protect the ACL against excessive rotation or extension. In some embodiments, the brace 105 is a closed-loop system that provides electrical muscle stimulation (EMS) based on feedback received from the brace 105. In some embodiments, the feedback is based on the applied EMS and the knee's response to the EMS. In some embodiments, the response to the EMS is measure by the resistance monitoring system. In some embodiments, the response to the EMS system is measured by a combination of the resistance and ultrasonic monitoring system.

In some embodiments, the brace 105 includes a proximal end 120 and a distal end 125. In some embodiments, the proximal end 120 is typically in physical contact with the person's femur. In some embodiments, the distal end 125 is typically in physical contact with the person's tibia. In some embodiments, the brace 105 is shown as having an opening at the knee 115. Although shown with an opening, the brace 105 can alternatively be closed at the knee 115 according to some embodiments.

In some embodiments, the proximal end 120 and distal end 125 of the brace 105 are connected by a pivotal joint or hinge 130. In some embodiments, the pivotal joint 130 enables the brace 105 to flex at the joint 130 when the person bends his or her knee 115. As described in more detail below, in some embodiments the pivotal joint 130 includes a digital positional encoder 735 which determines an absolute position of the knee 115. In some embodiments, the positional encoder 735 can provide this position of the knee 775 to the brace 105 digitally as part of the feedback in order for the brace 105 to record the position (or, in another embodiment, adjust) based on the transmitted position. Although the brace 105 is shown with one pivotal joint 130, in some embodiments the brace 105 can also include a second pivotal joint on the other side of the brace 105 which connects the other side of the proximal end 120 to the other side of the distal end 725. In some embodiments, brace 105 can be made from any of a variety of materials, such as from combinations of metal, foam, plastic, elastic material, composites, and straps. In some embodiments, the brace 105 can be secured to the person's body via one or more connectors 140, 150. In some embodiments, connectors 140, 150 are straps that connect to the brace 105 or to the respective connector 140, 150 itself Although shown with two connectors 140, 150, in some embodiments any number of connectors may be used. Connectors 140, 150 may be bolts, screws, pins, hook and loop fasteners, strings, clamps, or any other conventional connectors according to some embodiments.

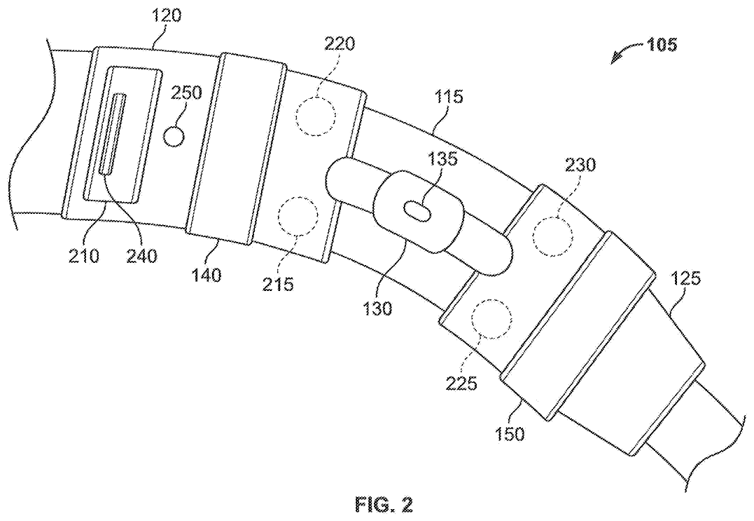

Also referring to FIG. 2, brace 105 includes control electronics 210 attached to or embedded within the brace 105 according to some embodiments. Although shown as being located in the proximal end 120 of the brace 105, in some embodiments control electronics 210 can be embedded within any location of the brace 105, such as within the distal end 725 of the brace 105, within the pivotal joint 130, and/or within one or more of the connectors 140, 150. Further, in some embodiments the control electronics 210 can be attached to the brace 105 via one or more cables or wires. In some embodiments, one or more of the components of the control electronics 210 is removable from the brace 105.

In some embodiments, the control electronics 210 enable EMS of one or more muscles that are in contact with the brace 105. Specifically, the brace 105 includes one or more sensors/pads/electrodes (e.g., sensor 275, 220, 225, 230) positioned in specific locations throughout the brace 105 according to some embodiments. Although the brace 105 shown in FIG. 2 includes two sensors 215, 220 positioned in the proximal end 120 of the brace 105 and two sensors 225, 230 positioned in the distal end 125 of the brace 105, in some embodiments the sensors 215, 220, 225, 230 can be in any configuration at any location. Further, although brace 105 is shown with four sensors 215, 220, 225, 230, any number of sensors (e.g., six sensors) can be used according to some embodiments. Additionally, in some embodiments the sensors 215, 220, 225, 230 may be any shape and any size, such as a circular shape or an oval shape. Additionally, in some embodiments, the sensors 215, 220, 225, 230 may be moveable (e.g., positioned in the brace but moveable by the doctor or patient). For example, the sensors 215, 220, 225, 230 can be moved within a circle/diameter of approximately 3 inches according to some embodiments. In some embodiments, the sensors 215, 220, 225, 230 are moveable but are secured with a strong Velcro material. In some embodiments, the sensors are electrodes or electrical contacts that can transmit and/or respond to voltage, current, and/or power. In some embodiments, the sensors are passive—they do not include an amplifier or any processing means.

In some embodiments, sensors around the knee include the following positions: 1) The motor point of the vastus medialis oblique, 2) The motor point of the vastus lateralis, and 3) the motor point of the distal central hamstring.

In some embodiments, the sensors 215, 220, 225, 230 are located on the interior wall of the brace 105 so that the sensors 215, 220, 225, 230 come in contact with the person's skin. In some embodiments, each sensor 215, 220, 225, 230 can take a power dissipation reading on the person's human tissues to determine how much the control electronics 210 “shocks” the person (i.e., how much current or voltage or power the sensors 215, 220, 225, 230 produce/apply to the person's human tissues). In some embodiments, galvanic skin resistance can be determined from the power dissipation reading. In some embodiments, the majority of the human body's resistance is in the skin—the dead, dry cells of the epidermis (the skin's outer layer) are usually poor conductors. Depending on the person, the resistance of dry skin is usually between 1,000-100,000 Ohms according to some embodiments. In some embodiments, the skin's resistance is lower if the skin is wet with an electrolytic solution (e.g., from sweat or from moisture). In some embodiments, conventional sensors apply a constant current to a person's skin based on an assumption of 500 Ohms of resistance for the person's skin. Unlike conventional sensors, in some embodiments, the sensors 215, 220, 225, 230 of the brace 105 measure the power dissipation of the human tissues of the person and adjust the output current/voltage/power based on this measurement. Thus, in some embodiments, the quantity of electricity output by one or more of the sensors 215, 220, 225, 230 is based on an electrical reading of the person's human tissues. In some embodiments, the reading occurs when the person's skin creates a closed circuit across two sensors (e.g., sensors 215, 220 or sensors 225, 230). For example, in some embodiments, when a person wears the brace 105, the person's skin on his or her leg closes the circuit between sensor 215 and sensor 220, thereby enabling a power factor reading to occur. In some embodiments, once this reading is transmitted to the control electronics 210, the electronics 210 adjusts the current/voltage/power output produced by the sensors to stimulate the muscles in the person's leg. In some embodiments, the sensors 215, 220, 225, 230 measure the patient's power dissipation factor periodically after a predetermined time period has elapsed (e.g., every 5 ms). In some embodiments, a medical professional can instruct the control electronics 210 to take a reading at a certain time or for a given amount of time (e.g., measure power dissipation every 5 ms from 6 PM to 7 PM). In some embodiments, the medical professional or the brace 105 itself can also be programmed to “shock” the patient at a predetermined time or times or on a specific schedule.

Further, conventional sensors or pads typically require the use of an electrolytic gel to facilitate conduction of the current/voltage/power output by the pads. Unlike conventional sensors, the sensors 215, 220, 225, 230 in some embodiments are not used with gel. Instead, in some embodiments the sensors 215, 220, 225, 230 are conductive silicon material that creates an electrical connection with a person's human tissues (e.g., via sweat, moisture, or skin itself). In some embodiments, the sensors 215, 220, 225, 230 are silicon with a conductive material (e.g., a metal) impregnated into the silicon, such as silicon nickel. Other conductive materials may be used, such as aluminum and/or carbon particles according to some embodiments. In some embodiments, the electrode pad is a carbon filled silicone sheet from Stockwell elastomerics, part No. SE 65-CON.

Many conventional pads stick to the patient's skin in order to make adequate contact with the skin. This causes problems, such as that the stickiness of the pad will cause hair or skin to be removed when the pad is removed or moved (e.g., as the brace moves or bends). Unlike these conventional sensors, in some embodiments, sensors 215, 220, 225, 230 do not use any sticky substance to connect to the patient's skin. Instead, in some embodiments the sensors 215, 220, 225, 230 can make physical contact with the human tissues (e.g., skin) via the placement of the sensors 215, 220, 225, 230 in the brace 105. In some embodiments, the sensors 215, 220, 225, 230 are used with gel. In some embodiments, the system can run both types of pads—pads with gel and pads without gel.

In some embodiments, the control electronics 210 receives feedback from one or more of the sensors 215, 220, 225, 230 and/or the positional encoder 135, thereby forming a closed loop system. Specifically, the brace 105 delivers EMS to the muscle via one or more of the sensors 215, 220, 225, 230 and adjusts the amount of current/voltage/power delivered by one or more of the sensors 215, 220, 225, 230 based on the readings obtained by the sensors 215, 220, 225, 230 and communicated to the control electronics 210 according to some embodiments.

In some embodiments, the control electronics 210 includes a microprocessor (e.g., ARM® CORTEX™ microprocessor developed by ARM® Ltd. of San Jose, Calif.) with one or more batteries and a communications module such as a Bluetooth® transceiver/module. In some embodiments, the control electronics 210 can provide stimulation via the sensors 215, 220, 225, 230 via one or more of the following types of waveforms or signals: parabolic arc (e.g., start soft and progressively increase), sine wave, cosine wave, pulse width modulation (PWM), pulse density modulation (PDM), square wave, sawtooth wave, and/or any conventional waveform. Further, in some embodiments, the control electronics 210 is configured to provide waveforms with any pulse duration and any pulse width.

In some embodiments, the sensor and the electrical stimulation electrode share a common contact point. In some embodiments, a MOSFET is included to build a switch between two phases. In some embodiments, one phase is completely isolated from the other phase. As a result of that isolation combined with knowing how much energy has been put into the system, an accurate reading of the power dissipation can be obtained according to some embodiments. In some embodiments, to determine when to input a sensing pulse versus when to input a stimulation pulse, using the known stimulation pulse value stored by the system as a reference, the control electronics 210 inputs a sense pulse with a higher voltage. In some embodiments, because a higher voltage was input in the sense pulse, any residual voltage from the stimulation phase doesn't matter because the voltage has been raised up to a new level to perform the sensing phase. Thus, in some embodiments, before taking a reading of the power dissipation, the voltage is automatically raised. If the voltage was not raised, then residual voltage would be obtained/read from the stimulation pulse according to some embodiments. In some embodiments, this therefore eliminates dealing with the residual voltage. In some embodiments, this is how the control electronics 210 gets around the capacitance and voltage in tissue. In some embodiments, the control electronics 210 raises the voltage of the entire area, and eliminates the problem of residual voltage and can then determine power dissipation.

In some embodiments, the control electronics 210 adjusts the current/voltage/power delivered to the sensors 215, 220, 225, 230 based on feedback from the positional encoder 735 and/or the sensors 215, 220, 225, 230. In some embodiments, one or more of the sensors 215, 220, 225, 230 behave differently depending on the position of the knee. Additionally, in some embodiments, the power loss varies for every person and changes during the course of operation, and the control electronics 210 can repeatedly measure the power dissipation of the patient via the sensors 215, 220, 225, 230 and repeatedly adjust the output current/voltage/power based on these readings. Thus, in some embodiments, a medical professional may set the brace to level 3 stimulation for person A because person A has sensitive skin, and may set the brace to level 6 stimulation for person B because person B has “thick” skin and is not as sensitive to the stimulation. In some embodiments, the level stimulation is set automatically based on the feedback. In some embodiments, the patient sets the level stimulation via a knob or control on the brace 105.

In some embodiments, the signals input by the control electronics 210 are constant current signals with variable voltage to attempt to maintain constant power output. In some embodiments, the current and/or voltage is varied to deliver substantially constant power. In some embodiments, the control electronics 210 inputs a test signal first (e.g., 200 volts) (e.g., sense pulse identified above) to break down the dielectric constant of the human tissues before inputting each stimulation signal. In some embodiments, this test signal creates an ionized channel or a channel of higher conduction. In some embodiments, after the test pulse is input into the human tissues, the stimulation pulse is input into the human tissues, which enables the stimulation pulse to have a lower voltage and therefore a lower total power. In some embodiments, the stimulation pulse is adjusted based on the readings from the test pulse. Thus, in some embodiments, the control electronics 210 measures the power dissipation before every stimulation pulse. This test pulse is why, if an electrical open circuit is detected or an electrical short is detected (e.g., if the patient falls into water), the stimulation pulse does not fire according to some embodiments. In some embodiments, this feature improves safety.

As described in more detail below, in some embodiments, the brace 105 may communicate data generated by the control electronics 210 and/or the feedback provided by the sensors 215, 220, 225, 230 and/or the positional encoder 735 to a medical professional (e.g., doctor, surgeon, and/or physical therapist). In some embodiments, the medical professional may adjust the brace 105 based on this data. For example, in some embodiments, the brace 105 may measure how strong the muscles surrounding the knee 115 are getting based on the EMS and/or the range of motion of the knee 115 (obtained via the positional encoder 135). As described in more detail below, in some embodiments, the medical professional can utilize this feedback and data to adjust the treatment of the patient. For example, in some embodiments, the medical professional may adjust the brace 105 based on these readings. Thus, in some embodiments, brace 105 provides a combination of bracing a joint and simultaneously stimulating the muscle(s) around the joint.

Additionally, in some embodiments, athletes or coaches may be interested in statistics produced by the control electronics 210, such as determining how much an athlete's joint can move after an injury or during recovery. As a specific example, a pitching coach on a baseball team is likely interested in statistics associated with a pitcher's movement of his pitching arm according to some embodiments.

In some embodiments, the control electronics 210 includes one or more control programs that a medical professional or patient can select and/or program. In some embodiments, the control programs are configured to be dynamic (e.g., changeable or variable, not a fixed frequency, not fixed timing, not a fixed waveform, etc.) and are configured to cause different types of EMS to be executed on different parts of the patient's body. For example, if the feedback data from the control electronics 210 indicates that the patient's vastus medialis oblique muscles are getting stronger while the patient's distal central hamstring (or, in another embodiment, the patient's calf muscle) is not getting stronger, a medical professional (e.g., doctor or physical therapist) may instruct, via one or more of these programs, the brace 105 to execute a predetermined control program according to some embodiments. In some embodiments, this predetermined control program is configured to cause sensors 215, 220 to output a current of 7 mA of DC current for 30 seconds and then 5 mA for 20 seconds. In some embodiments, the predetermined control program is configured to cause sensors 225, 230 to output a current of 1 mA for 50 seconds, thereby providing significantly more stimulation to the patient's vastus medialis oblique muscles compared with the patient's distal central hamstring (or, in some embodiments, the patient's calf muscle). In some embodiments, the brace 105 includes specific programs for the first week after surgery, specific programs for the first month after surgery, specific programs for arthritis, etc.

In some embodiments, the brace 105 includes an authentication button 250. In some embodiments, the authentication button 250 is a button that has to be pressed by the patient in order for a program to execute. Thus, in some embodiments, the authentication button 250 is a security feature of the brace 105—the brace 105 cannot be compromised or caused to execute one or more stimulation programs or actions until the wearer of the brace presses the authentication button 250. For example, if a medical professional remotely accesses the control electronics 210 and attempts to have the brace 105 execute specific muscle stimulation or adjust the range of motion of the brace 105 for the patient, the brace 105 will not execute the stimulation or adjust the range of motion until the patient presses the authentication button 250 according to some embodiments.

In some embodiments, the control electronics 210 may also include a display 240. In some embodiments, the display 240 may display statistics associated with the brace, such as how much power dissipation the sensors 215, 220, 225, 230 are measuring, how much current/voltage/power the sensors 215, 220, 225, 230 are delivering, the angle of the positional encoder 135, programs executing or past programs executed, the date, the time, the patient's next appointment (e.g., with a doctor or a physical therapist), average range of motion of the joint over a fixed period of time or any other information associated with the brace 105. In some embodiments, the control electronics 210 includes a keyboard to enable the user to provide input to brace 105.

In some embodiments, the brace 105 may also have visual feedback. For example, one or more LEDs can be located on the brace 105 for alerting the patient of a specific occurrence according to some embodiments. For instance, in some embodiments, an LED is configured to light when the brace 105 is waiting for the patient to press the authentication button 250.

Additionally, in some embodiments, the brace 105 is configured to transmit the generated data (feedback data) to a computing device associated with, for example, the user or the medical professional. Due to the communication of the brace 105 with the computing device, the medical professional can be notified or will see that the patient is not wearing the brace if an electrical open circuit is detected according to some embodiments. Similarly, in some embodiments, if the patient falls into a pool, the medical professional will know this as well because an electrical short is detected. In some embodiments, the medical professional or brace 105 is configured to transmit the data generated by the brace 105 to an insurance company. In some embodiments, the insurance company can then determine, from this data, whether the patient is performing his or her exercises, is wearing the brace throughout the day, etc. In some embodiments, this may affect the insurance provided by the insurance company (e.g., lower premium if patient wears the brace all day and is doing exercises). In some embodiments, medical professionals such as doctors may request or obtain a specific insurance reimbursement when prescribing the brace. In some embodiments, a specific insurance code may be available to the medical professional for prescribing the brace.

In some embodiments, the brace 105 is an unloader brace. In some embodiments, unloader braces are usually prescribed for people who have medial (inner part of the knee) compartment knee osteoarthritis. In some embodiments, these knee braces str configured to unload stress from the affected joint by placing pressure on the thigh bone. In some embodiments, this forces the knee to bend away from the painful area. Thus, in some embodiments, an unloader brace is a brace that is stronger and more rigid on one part of the knee. In some embodiments, brace 105 exerts a force on one direction of the knee. In some embodiments, an adapter piece attaches to the brace 105 to exert such a force, thereby forming an unloader brace.

In some embodiments, the brace 105 may also be configured to provide co-coupled contraction of different muscle groups. For example, in some embodiments, four sensors (e.g., including sensors 215 and 220) can be located on the quadriceps muscles and two sensors (e.g., sensors 225 and 230) can be located on the hamstring muscles. In some embodiments, the brace 105 can stimulate both sets of muscles at different times or simultaneously, such as at the same or at different frequencies, patterns, and/or waveforms. For example, when the brace 105 activates or fires the sensors 215, 220 at a first rate, the brace 105 is configured to activate or fire the sensors 225, 230 at a second, slower rate (or, in another embodiment, at the same rate) according to some embodiments. In some embodiments, the firing of the hamstring at a different frequency than (or at the same time as) the quadriceps muscles results in co-coupled contraction. In some embodiments, the firing of the hamstring (the antagonistic muscle group) with the quadriceps muscles results in the strengthening of both sets of muscles. In some embodiments, the stimulation of the antagonistic muscle group strengthens both sets of muscles, even when only one of the muscle groups is atrophied. In some embodiments, the brace 105 is configured to execute a first program for a first muscle and execute a second program for a second, antagonistic muscle. In some embodiments, the doctor positions the sensors 215, 220, 225, 230 on the brace 105 for this co-coupled contraction to occur. In some embodiments, the sensors 215, 220, 225, 230 are integrally positioned within the brace 105 to cause the co-coupled contraction of different muscle groups.

In some embodiments, the brace 105 includes a data gathering thermometer which can determine the temperature of the patient and adjust one or more of the sensors 215, 220, 225, 230 and/or the control electronics 210 based on this temperature.

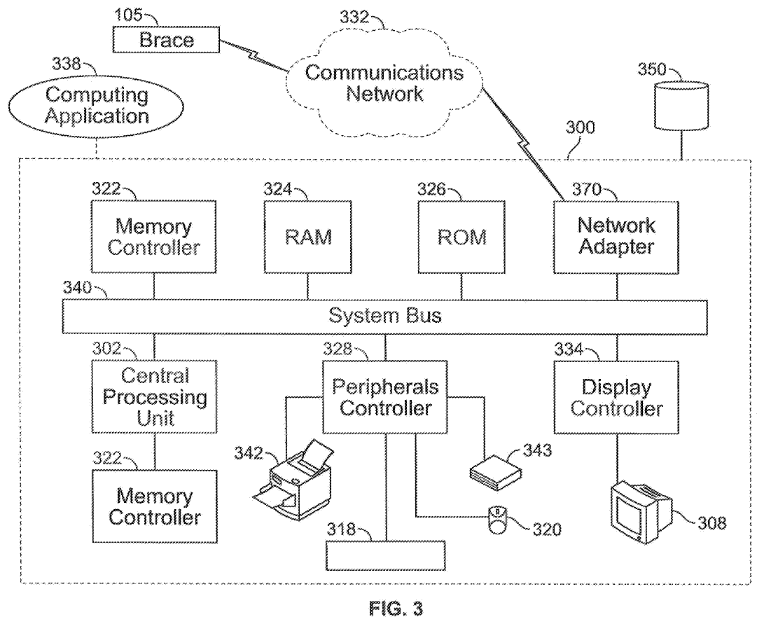

FIG. 3 shows a computer 300 for controlling one or more components of the system according to some embodiments. In some embodiments, the system control electronics 210 is configured to communicate wirelessly and/or via a wired connection with a computing device 300. In some embodiments, examples of the computer 300 include, but are not limited to, personal computers, digital assistants, personal digital assistants, mobile phones, smartphones, tablets, or laptop computers. In some embodiments, the computer 300 is the patient's device or a device associated with a medical professional. In some embodiments, the computer 300 is configured to enable a medical professional to retrieve and analyze data transmitted from the system. In some embodiments, the system is configured to transmit data in real-time, so that the medical professional can analyze the data and/or adjust one or more settings at any time.

In some embodiments, the computer 300 is configured to read instructions from media and/or a network port. In some embodiments, the computer 300 is configured to connect to the Internet or an intranet. The computer 300 includes one or more processors 302, one or more non-transitory computer readable storage media (e.g., RAM 324 and/or ROM 326), one or more input devices (e.g., keyboard 318, printer 342, external memory 343, and/or monitor 308) In some embodiments, computer 300 is in communication with and/or is a server computer. The computer 300 includes any suitable conventional means of transmitting and/or receiving data. For example, in some embodiments the computer 300 includes a network connection, a wireless connection or an internet connection for transmitting and/or receiving data to/from the garment system.

In some embodiments, the computer 300 is configured to execute a variety of computing applications 338, including computing applications (i.e., Apps), computer programs, and/or other instructions for computer 300 to perform at least one function, operation, and/or procedure. In some embodiments, computer 300 is configured to read non-transitory storage media on which include instructions in the form of software. In some embodiments, the computer readable storage media is configured to tangibly store computer readable instructions that when executed by the one or more processors cause the computer 300 to perform one or more functions described herein.

As will be appreciated by those skilled in the art, in some embodiments, a computer readable medium stores computer data, includes computer program code that is executable by a computer, in machine readable form. In some embodiments, by way of example, and not limitation, a computer readable medium may comprise computer readable storage media, for tangible or fixed storage of data, or communication media or for transient interpretation of code-containing signals. In some embodiments, computer readable storage media, as used herein, refers to physical or tangible storage (as opposed to signals) and includes without limitation volatile and non-volatile, removable and non-removable storage media implemented in any conventional method or technology for the tangible storage of information such as computer-readable instructions, data structures, program modules or other data according to some embodiments. In some embodiments, computer readable storage media includes, but is not limited to, RAM, ROM, EPROM, EEPROM, flash memory or other solid state memory technology, CD-ROM, DVD, and/or other optical storage, magnetic cassettes, magnetic tape, magnetic disk storage or other magnetic storage devices, and/or any other physical or material medium which can be used to tangibly store the desired information or data or instructions and which can be accessed by one or more computers and/or processors.

In some embodiments, the CPU 302 fetches, decodes, and executes instructions, and transfers information to and from other resources via the computer's main data-transfer path, system bus 340. In some embodiments, system bus 340 connects the components in the computer 300 and defines the medium for data exchange. In some embodiments, access to the RAM 324 and/or ROM 326 may be controlled by memory controller 322. In some embodiments, the memory controller 322 is configured to provide an address translation function that translates virtual addresses into physical addresses as instructions are executed. In addition, according to some embodiments, the computer 300 includes at least one peripheral controller 328 responsible for communicating instructions from the CPU 302 to peripherals, such as, printer 342, keyboard 318, mouse 320, and/or data storage drive 343. In some embodiments, display 308, which is controlled by display controller 334, is used to display visual output generated by the computing device 300. In some embodiments, the visual output may include text, graphics, animated graphics, video, and/or or a graphical user interface (GUI). In some embodiments, the display controller 334 includes electronic components required to generate a video signal that is sent to display 308. Further, according to some embodiments, the computer 300 contains network adaptor 336 which is configured to connect the computing device 300 to an external communications network 332. In some embodiments, Bluetooth® products may be used to provide links between brace 105 and mobile computers, mobile phones, portable handheld devices, personal digital assistants (PDAs), tablets, and other mobile devices and connectivity to the Internet. Bluetooth® is a computing and telecommunications industry specification that details how mobile devices can easily interconnect with each other and with non-mobile devices using a short-range wireless connection according to some embodiments.

In some embodiments, computer 300 is configured to utilize a specific application 338 (also referred to as an “app”) to communicate with and/or program the garment system. In some embodiments, the computer 300 downloads the app 338 from the communications network 332 (e.g., from an “app store” on the Internet). In some embodiments, the app 338 is configured to calculate and/or provide statistics, graphs, normalized data, raw data, averages (e.g., average flexion and average extension), real-time data, etc. to a user and/or medical professional. In some embodiments, the app 338 provides output data that is in a format customizable by the user or medical professional. In some embodiments, the app 338 is configured to communicate with other programs, such as hospital software, word processing software (e.g., Microsoft Word®), spreadsheet software (e.g., Microsoft Excel®), email software (e.g., Microsoft Outlook®), publishing software (e.g., Microsoft Powerpoint®), etc. to further analyze or display the data. In some embodiments, the app 338 is configured to provide a graphical user interface (GUI) or a text-based user interface. In some embodiments, the app 338 is configured to communicate with the garment system and/or a database (as described below) to display and analyze the data generated by the garment system and/or provided by a user and/or doctor. In some embodiments, the app 338 is configured to execute instructions and/or sequences created by the user and/or medical professional at a location remote from the garment system. In some embodiments, as described above, the patient has to press the authentication button 250 in order for the brace 105 to actually execute the program being set remotely.

PCT Application PCT/US2018/044124 (WO 2019/023598; hereafter PCT '124) is commonly owned by Applicant and the entire disclosure is hereby incorporated by reference into this application. PCT '124 describes various arrangements and components that are suitable to implement one or more aspects of the system and/or the computer 300 described herein. For example, the GUIs, therapy control systems, and/or garments, as non-limiting examples, described in PCT '124 are all compatible with one or more portions of the system and/or the computer 300, such as the occlusion and/or pelvic stimulation systems according to some embodiments. Furthermore, portions of the PCT '124 recited according to some embodiments are integratable to some all and/or some embodiments of the instant application. PCT '124 is incorporated by reference to provide additional enablement for those of ordinary skill to understand how to make and use the system and/or to distinguish over the prior art when describing the metes and bounds of some embodiments.

In some embodiments, the computer 300 is a portable data reader that is specifically associated with the brace 105. For example, a medical professional can synchronize the reader 300 with the patient's brace 105 when the medical professional provides the brace 105 to the patient according to some embodiments. In some embodiments, at a later time (e.g., at a subsequent visit), the medical professional can use the reader to capture data from the brace 105. In some embodiments, the medical professional can then use the reader to view the retrieved data (during the patient's visit and/or before the visit). In some embodiments, a user executes a browser to view digital content items and can connect to a server via a network, which is typically the Internet, but can also be any network, including but not limited to any combination of a LAN, a MAN, a WAN, a mobile, wired or wireless network, a private network, or a virtual private network.

In some embodiments, the computer 300 is in communication with a database 350. In some embodiments, the computer 300 may store data transmitted by the brace 105 in database 350. In some embodiments, the database 350 may be an internal database of the computer 300. In some embodiments, the database 350 may be an external database in communication with the computer 300. To protect patient confidentiality and to protect the security of the data, usage data that is transmitted from the devices (via Bluetooth, WiFi, or via other means) is encrypted to ensure that only the patient or the patient's doctor can obtain access to this medical information according to some embodiments. In some embodiments, the encryption is done via either software executing on the processor or via external hardware that processes the data before it is transmitted. In some embodiments, each set of logs is uniquely tied to the device that created them. In some embodiments, this can be done by the device tagging the data being transmitted from the device with a unique identifier associated with the device itself In some embodiments, the unique identifier is set either by the processor or by an external component of the system (e.g., UUID chip).

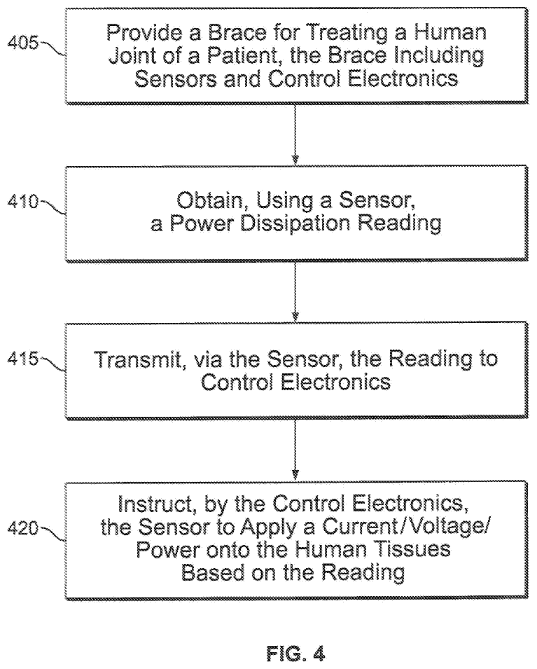

FIG. 4 shows a flowchart illustrating one or more steps performed in the closed loop feedback bracing system according to some embodiments. In some embodiments, a brace is provided for treating a human joint of a patient (e.g., knee, elbow, back, spine, wrist, etc.) (Step 405). In some embodiments, the brace includes sensors and control electronics. In some embodiments, one or more sensors 215, 220, 225, 230 obtain a power dissipation reading (Step 410). As described above, in some embodiments, two sensors obtain a power dissipation reading when skin completes the circuit between the two sensors. In some embodiments, the sensor or sensors 215, 220, 225, 230 then transmit the power dissipation reading to the control electronics 210 (Step 415). In some embodiments, the control electronics 210 instruct the sensor or sensors 215, 220, 225, 230 to apply a current/voltage/power onto the human tissues based on the power dissipation reading (Step 420). In some embodiments, this results in a closed loop feedback system, where the output of the brace 105 is dependent upon the input readings of power dissipation (e.g., of sweat, of human tissues, etc.). In some embodiments, the output of the brace 105 is dependent upon the input readings of power dissipation from the sensors 215, 220, 225, 230.

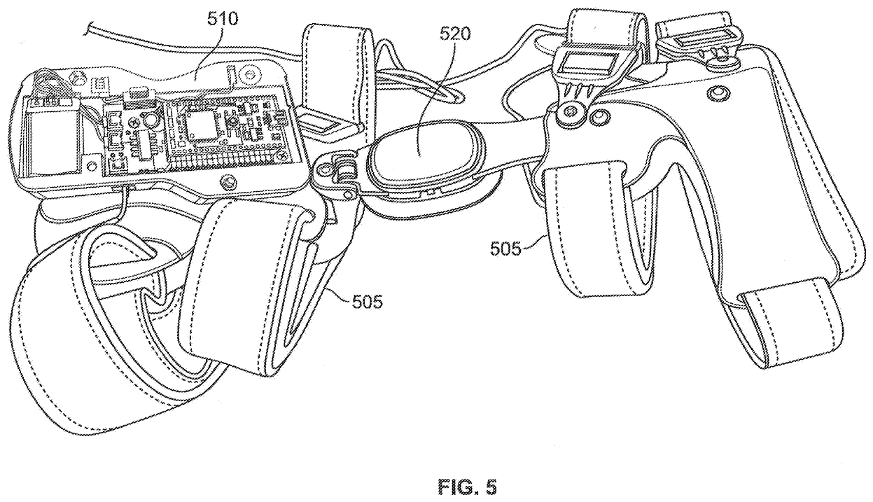

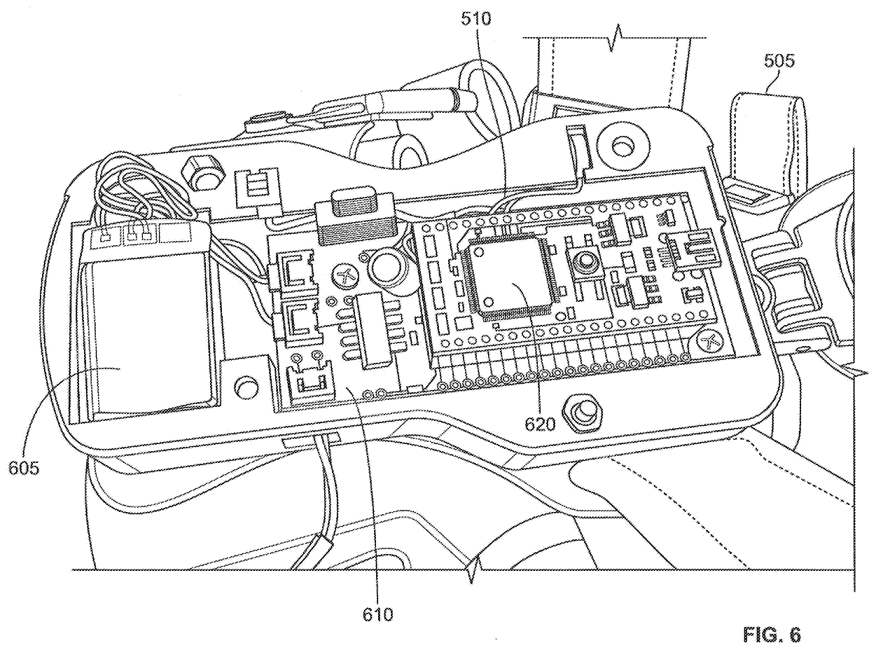

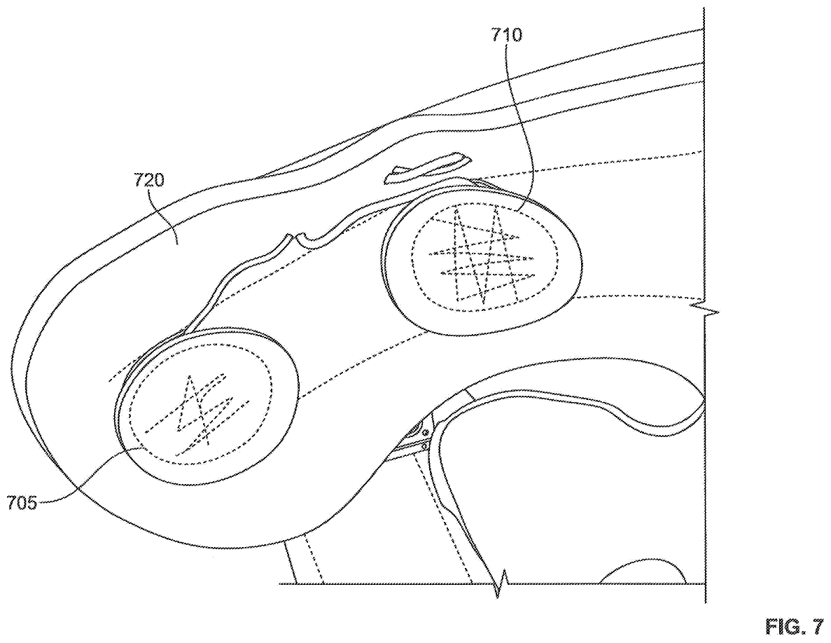

FIG. 5 is a perspective view of an embodiment of a knee brace 505 including control electronics 510 and a pivotal joint 520 according to some embodiments. FIG. 6 is a more detailed perspective view of control electronics 510 of the knee brace 505 according to some embodiments. In some embodiments, the control electronics 510 include a battery 605 connected to a circuit board 610. In some embodiments, the circuit board 610 includes a microprocessor 620 for the programming of and functioning of the brace 505. FIG. 7 is a perspective view of two sensors 705, 710 of the knee brace 720 according to some embodiments. In some embodiments, the sensors 705, 710 are located on the interior wall of the brace 720 so that the skin of the wearer of the brace is in physical contact with the sensors 705, 710.

In some embodiments, the brace enables the patient to move the joint (e.g., knee) while wearing the brace and while the sensors are providing EMS and obtaining the power dissipation of the patient's human tissues. In some embodiments, the brace can cross the joint (e.g., knee) and still enable motion by the patient because there is no sticky adhesive used with the sensors. Thus, in some embodiments, the brace is configured to provide EMS while the patient is doing physical therapy or exercising.

In some embodiments, a control unit connects to the brace and controls or is configured to program the brace. In some embodiments, one or more of the control electronics are located in the control unit and not in or on the brace. For example, in some embodiments, the control unit is configured to connect to (e.g., wirelessly or via one or more wires) and/or communicate with the sensors. In some embodiments, the control unit is configured to program the sensors to run specific programs, receive the power dissipation from the sensors, and/or adjust the EMS based on the received readings. In some embodiments, the brace includes a memory chip configured to store the program(s) associated with the specific brace, such as the waveforms applied to the brace at specific times. In some embodiments, when the control unit connects to the brace, the control unit is configured to read the program(s) from the memory chip on the brace and/or communicate with the sensors to run the read program. In some embodiments, the control unit is configured to read an identifier from the brace to identify the type of brace (e.g., knee brace, shoulder sling, sleeve, etc.). Thus, in some embodiments, the control unit is configured to be used with and communicate with any number of garments such as a sleeve, a wrap, a garment (e.g., shorts or compression shorts (CAM)), a brace, a sling, etc. In some embodiments, the garment can be for any body part, such as a knee, ankle, wrist, shoulder, back, calf, hip, thigh, elbow, etc. In some embodiments, the garment can be worn by the patient after surgery, during exercise, for arthritis, or any other time. In some embodiments, the garment can be rigid or flexible and is configured to be worn across a joint.

In some embodiments, the control unit is configured to connect with the garment via a plug or port located on the garment and/or connected to the garment. In some embodiments, once connected, the control unit is configured to determine the type of garment attached and execute the specific program for the specific garment via communication with the sensors on the garment. Thus, in some embodiments, the single control unit is configured be used with any soft garment(s) purchased or utilized by a patient. In some embodiments, the control unit is configured to communicate (e.g., wirelessly) with the medical professional (e.g., doctor) periodically, at set times, when the program(s) are executed, and/or any other time. In some embodiments, the control unit is a physical device (e.g., that the patient can clip onto their belt or, e.g., in a pocket in the garment). In some embodiments, the control unit is an “app” residing on a smartphone or computing device. In some embodiments, the control unit is configured to download data to a computing device for review and/or analysis. In some embodiments, the control unit has a display including a graphical user interface (GUI) that is configured to display one or more options to the user (e.g., medical professional or patient), such as to select the body part being supported, to select the program (e.g., waveform(s)) to execute, etc. In some embodiments, the control unit can be used to update the information stored on the soft garment, such as by downloading new programs into the soft garment for storage and future execution.

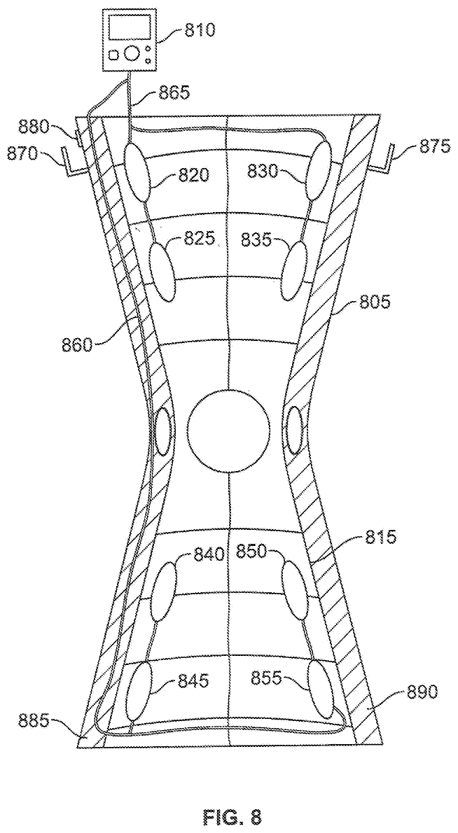

FIG. 8 is a perspective view of a garment 805 connected to control unit 810 according to some embodiments. In some embodiments, garment 805 is a “short brace” that includes a sleeve/wrap 815 that is part of the garment 805. In some embodiments, the sleeve/wrap 815 cannot be separated from the garment 805. In some embodiments, the sleeve/wrap 815 includes one or more sensors. In some embodiments, the sleeve/wrap 815 includes one or more of first upper sensor pair 820, 825; second upper sensor pair 830, 835; and/or a first lower sensor pair 840, 845 and a second lower sensor pair 850, 855. In some embodiments, the current flows between two connected sensors of a sensor pair, such as between sensor 820 and sensor 825. In some embodiments, the connected sensor pairs form a channel. In some embodiments, when one channel (e.g., between sensor 820 and sensor 825) is conducting current, the other channels (e.g., channel between sensors 830, 835) are floating and therefore no current is flowing between these other “floating” channels.

In some embodiments, photosets are used for high frequency isolation. Photofets facilitate noise isolation because there is an absorption band that minimizes high frequency noise for transitions between, for example, 0.01 and 0.1 milliseconds according to some embodiments. In some embodiments, anything above that frequency (above 10 kHz) is removed, and because the transistors (FETS) are operated well beyond linear transition states, the drive signals are clean with little slew and no backscatter exhibited on output electrodes. Thus, photoisolation is obtained according to some embodiments.

In some embodiments, the sensors 820, 825, 830, 835, 840, 845, 850, 855 are connected to the control unit 810 via wires 860, 865. In some embodiments, the sensors 820, 825, 830, 835, 840, 845, 850, 855 are in communication with the control unit wirelessly. In some embodiments, the garment 805 includes brackets 870, 875 for secure placement of the control unit 810. In some embodiments, the control unit 810 plugs into the garment 805 via port 880. In some embodiments, the garment 805 includes stays 885, 890.

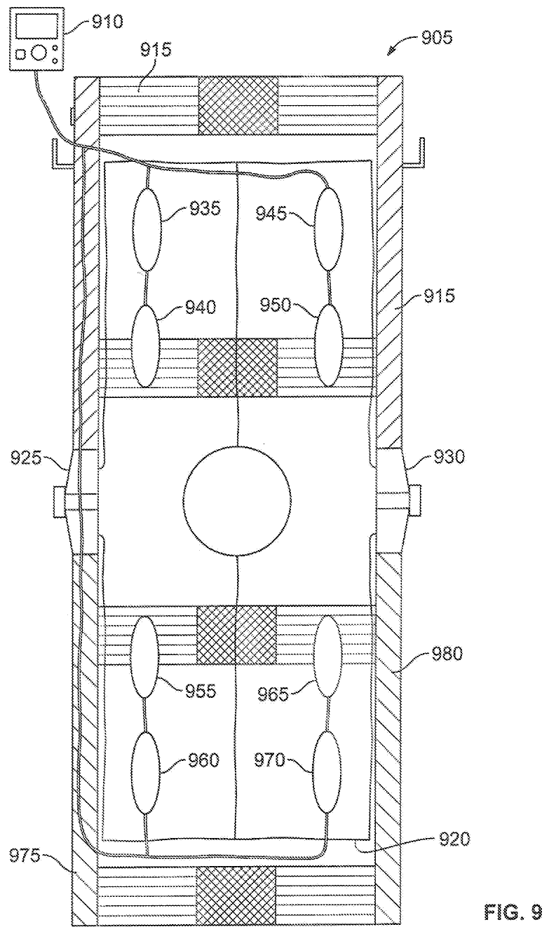

FIG. 9 is a perspective view of an embodiment of a garment 905 connected to control unit 910 according to some embodiments. In some embodiments, garment 905 is a “long brace” that includes a brace 915 and a sleeve/wrap 920 that is inside the brace 915. In some embodiments, the sleeve/wrap 920 is connected to the brace 915 at hinges 925, 930. In some embodiments, the hinges 925, 930 can be adjustable hinges, such as hinges that can adjust between 0°, 45°, 90°, and open. In some embodiments, the sleeve/wrap 920 can be separated from the brace 915. In some embodiments, the sleeve/wrap 920 includes one or more sensors, such as a first upper sensor pair 935, 940 and/or a second upper sensor pair 945, 950, and/or a first lower sensor pair 955, 960 and a second lower sensor pair 965, 970. As described above, in some embodiments, the current flows between two connected sensors of a sensor pair, such as between sensor 935 and sensor 940. In some embodiments, the connected sensor pairs form a channel. In some embodiments, when one channel (e.g., between sensor 935 and sensor 940) is conducting current, the other channels (e.g., channel between sensors 945, 950) are floating and therefore no current is flowing between these other “floating” channels. In some embodiments, the brace 915 includes stays 975, 980.

In some embodiments, the long brace 905 is a brace 915 with sleeve/wrap 920 that extends past the joint (e.g., knee). Thus, in some embodiments, unlike the short brace 805, which has an attached sleeve 815, the long brace 905 has a sleeve 920 that enables removal of the brace 915 from the sleeve 920.

In some embodiments, each sensor is packaged with moisturizer (e.g., a generic hand cream) applied thereon. In some embodiments, each sensor with moisturizer can have, for instance, a cellophane cover on the sensor and the patient or medical professional would remove the cellophane cover when the garment is removed from its package. In some embodiments, the sensor will sense how dry the patient's skin is and communicate this information to the control unit. In some embodiments, the control unit is configured to then provide a notification to the patient or medical professional that the patient's skin needs to be moisturized.

In some embodiments, the sleeve, brace, or garment provides support to the calf muscle of a patient and electrodes/sensors apply EMS to the calf muscle in a closed loop fashion as described. Thus, in some embodiments, the soft garment stimulates the calf muscle(s) to facilitate prevention of deep vein thrombosis (DVT).

In some embodiments, the garment can be a garment providing lumbar support. In some embodiments, the garment can cross the hip joint and can have electrodes on one or both sides of the hip joint while also providing back support. In some embodiments, the electrodes are placed around one or more of the hip, the lower back, and the legs.

In some embodiments, calf stimulation and quad stimulation typically require application of EMS with different amplitudes. Thus, in some embodiments, the closed loop system is configured to be used to monitor amplitude. In some embodiments, one sleeve is configured to treat different muscle groups and because monitoring reaction of muscle to stimulation and adjusting amplitude of pulse via the described closed loop system, one garment (e.g., sleeve) can be used in for different muscle groups according to some embodiments.

In some embodiments, the power dissipation of a short “sense pulse” is obtained before each stimulation pulse. In some embodiments, each stimulation pulse is adjusted based on one or more power dissipation measurements in order to maintain constant power output across each pulse. In some embodiments, each electrode used to provide the electrical stimulation contains a sensor so that the power dissipation is determined at the stimulation site.

In some embodiments, the closed loop provides several benefits. For example, if the measured power dissipation from the sensing pulse exceeds preset boundaries, the device will end its stimulation sequence before discharging the stimulation pulse according to some embodiments. As another benefit, in some embodiments, each sense pulse creates or maintains a conductive channel through the human tissues by exceeding the breakdown voltage of the human tissues. In some embodiments, the creation of this dielectric breakdown improves efficiency and safety by reducing the power required to contract a desired muscle with a given stimulation pulse. In some embodiments, by reducing the power requirements of the stimulation pulses and maintaining constant power across every stimulation pulse, the risk of painful shocks and skin burns is eliminated. Further, in some embodiments, the overall efficiency of the unit is dramatically improved, allowing for a reduction in size of the electrical components compared to existing units, making the brace more portable and easier to use.

In some embodiments, one advantage of applying constant power is avoiding the harmful effects of cellular damage. In some embodiments, a cell has a maximum wattage it can survive. In some embodiments, after overcoming the dielectric constant, conventional units may introduce cellular damage. In some embodiments, once the dielectric constant is overcome, milliwatts of power are needed. Thus, once the dielectric breakdown occurs and current is flowing, the control electronics 210 reduces the power to a fixed, low power that can be adjusted by the user according to some embodiments.

In some embodiments, once power dissipation is determined, the power to pump into the human tissues can be determined after the conductive channel is created. In some embodiments, the channel is maintained, and can determine characteristics of the channel (e.g., power received and power transmitted). Thus, in some embodiments, power dissipation can be determined.

In some embodiments, the device self-tunes it's electrical output by modifying the drive voltage of the HV power supply in order to maintain the desired output power (e.g., in watts). In some embodiments, the required power output is calculated by measuring the power dissipation of the electrical circuit formed by the electrodes and the human tissues and applying one or more algorithms to the power dissipation measurement and the desired waveform data.

In some embodiments, in order to achieve this, the output of a flyback mode switching power supply is modified to generate a stable, regulated DC. In some embodiments, by taking this approach rather than the traditional approach of a push-pull driver against a transformer, the system is configured to provide a clean DC signal, rather than a noisy signal with potential high frequency A/C. In some embodiments, this is essential for accurate measurement, and/or true closed loop operation.

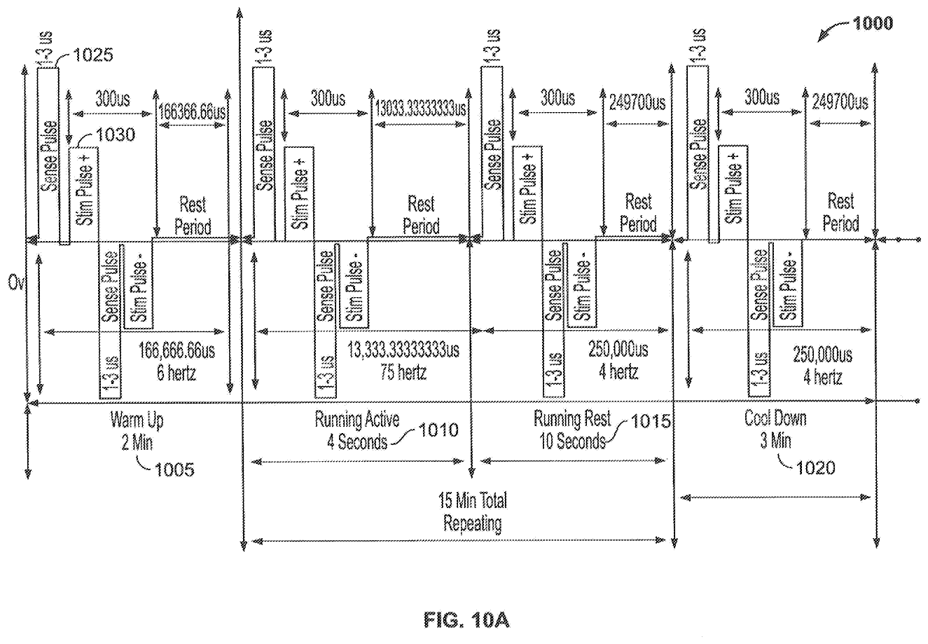

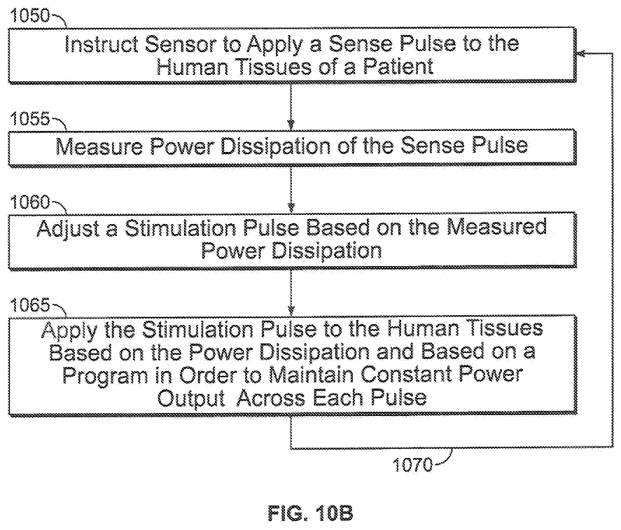

In some embodiments, the power dissipation is measured before every stimulation pulse and the stimulation pulse is adjusted to maintain a constant power output for each pulse. Referring now to FIG. 10A, the DC signals 1000 transmitted into the human tissues by the electrodes/sensors are shown according to some embodiments. In some embodiments, the signals 1000 include a warm up phase 1005, a running active phase 1010, a running rest phase 1015, and a cool down phase 1020. In some embodiments, each phase includes a sense pulse (referred to hereinafter as sense pulse 1025), which is a short pulse to overcome the dielectric constant of the human tissues (to create an ion channel in the human tissues so that current can flow), and/or to sense the power loss in the circuit to determine how much power in a stimulation pulse should be applied (and/or to determine whether it is safe to transmit the stimulation pulse, as described in more detail below). In some embodiments, the sense pulse 1025 in one embodiment is approximately 10-180 V and lasts 1-3 μs. In some embodiments, after the sense pulse is transmitted, the sensor typically transmits a stimulation pulse (hereinafter stimulation pulse 1030). In some embodiments, the stimulation pulse 1030 is, in one embodiment, approximately 18-20V and typically in the range of 1 μs to 200 μs. Thus, after the sense pulse 1025, the voltage drops significantly to limit the current according to some embodiments. In some embodiments, the power dissipation is then measured before the introduction of the next stimulation pulse 1030. In some embodiments, the power transmitted is dissipated before the change in polarity of the signals, thereby preventing charge transfer during zero crossing, ensuring the signal remains purely DC.

In some embodiments, there is a gap in time between the end of the sense pulse 1025 and the start of the stimulation pulse 1030. In some embodiments, the pulses then switch polarity. In some embodiments, before every stimulation pulse 1030, the sensor transmits a sense pulse 1025 to determine how much power has been dissipated and whether it is safe to deliver the stimulation pulse 1030. In some embodiments, the signals produced after the sense pulse introduce a very small power factor, on the order of milliwatts.

FIG. 10B is a flowchart showing an embodiment of steps performed by the control electronics according to some embodiments. In some embodiments, the control electronics instructs a sensor in a sensor pair to apply the sense pulse 1025 to the human tissues of a patient (Step 1050). In some embodiments, the sensor (or other sensor in the sensor pair) measures the power dissipation of the sense pulse 1025 in the human tissues (Step 1055). In some embodiments, the control electronics adjusts the stimulation pulse 1030 based on the measured power dissipation (Step 1060). In some embodiments, the control electronics then instructs the sensor to apply the stimulation pulse 1030 to the human tissues based on the power dissipation and based on the program in the garment in order to maintain constant power output across each pulse. In some embodiments, Steps 1050-1065 are repeated (Step 1070).

Referring to FIG. 11, the other sensor in the pair of sensors (per sensor channel) provides the return path for the electrical current from the transmission of the stimulation pulses 1030 according to some embodiments. In some embodiments, the sense pulse 1025 measures how long it takes to receive a return pulse 1105 on the receiving electrode side. In some embodiments, if the sensor determines the propagation delay between sent pulse (e.g., pulse 1025) and return pulse 1105, the sensor (or control electronics 210) can determine the maximum stimulation pulse 1030 to apply. In some embodiments, the return pulse 1105 is typically a square pulse.

As shown in FIG. 11, the propagation delay 1110 between the stimulation pulse 1030 and the receive pulse 1105 is the time difference between the start time of the stimulation pulse 1030 and the start time of the receive pulse 1105 according to some embodiments. In some embodiments, the change in distortion 1120 is the difference between the pulse widths of the two pulses 1030, 1105. In some embodiments, the change in distortion is used to determine whether the muscle is being charged as an inductor and whether the muscle is storing power. In some embodiments, the change in distortion is for calibrating the algorithm and another point of feedback. In some embodiments, the gel applied with the sensors (e.g., hydrogel) is introducing (or increasing) the propagation delay.

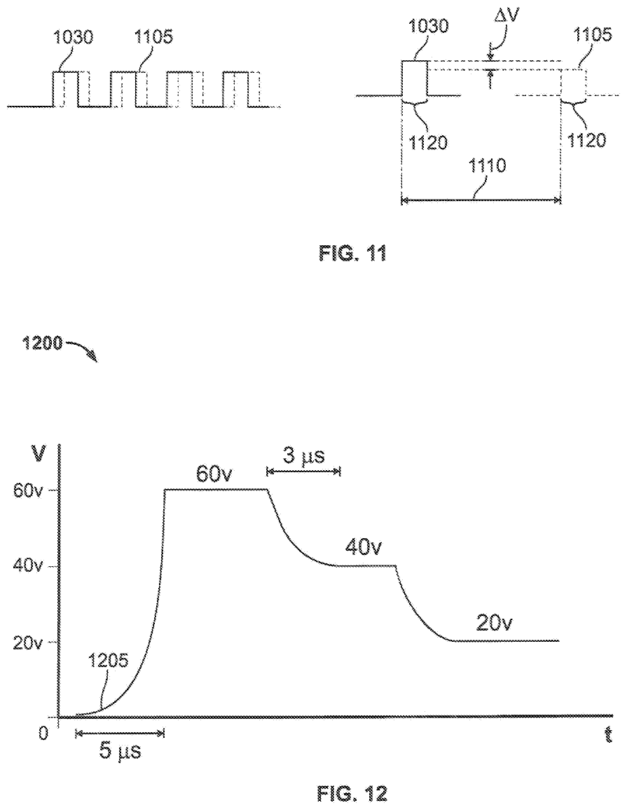

FIG. 12 shows an embodiment of a power supply signal 1200 produced by the power supply (as described in more detail below) according to some embodiments. In some embodiments, the power supply signal 1200 includes a ramp up phase 1205 that typically lasts 5 μs. In some embodiments, the voltage peaks at 60 V, and then drops down after a period of time to 40 V. In some embodiments, the voltage signal then drops down, after a second period of time, to 20 V. In some embodiments, the power supply is a voltage-controlled power supply and/or a current controlled power supply. In some embodiments, when an increase in current (or voltage) is needed, the power is increased.

In some embodiments, the conventional power supplies used with electrical stimulation for braces or wearable components typically utilize multiple pulses (power generation and switching technology). In some embodiments, they often generate a 24 V supply and then have a transformer, H-bridge, and produce a pulse train (with a ripple), where the transformer averages the signal out (e.g., 1:10 or 1:20 ratio). In some embodiments, unlike these conventional systems, the power supply here provides a steady signal with a small ramp up phase, which enables the closed loop system.

In some embodiments, the output is an analog voltage upon which current is clamped. In some embodiments, this power supply enables precise and accurate, virtually noise-free measurements. In some embodiments, the conventional power supplies induce current flow based on pulse-width modulation (PWM). In some embodiments, PWM systems do not enable precise and accurate measurements due to the noise introduced from PWM and due to field saturation of their transformer(s). Further, in some embodiments, the power supply in this system enables a wide range of waveforms and protocols to be run based on the information stored on the soft garment. Additionally, in some embodiments, if it is determined that a protocol is harmful and cannot be run (e.g., determined by the FDA), this power supply enables the system to be operational much faster than others because only the soft garment needs to be changed.

In some embodiments, 0-3.3 V input voltage controls the output across the full targeted range of the power supply. Thus, in some embodiments, to generate the 60 V output maximum range, 3.3 V is provided as reference input to the power supply. In some embodiments, a dedicated voltage-controlled power supply is present per channel, which means there is no time division. In some embodiments, conventional power supplies use time division to supply power to multiple electrodes. In some embodiments, there is no time division.

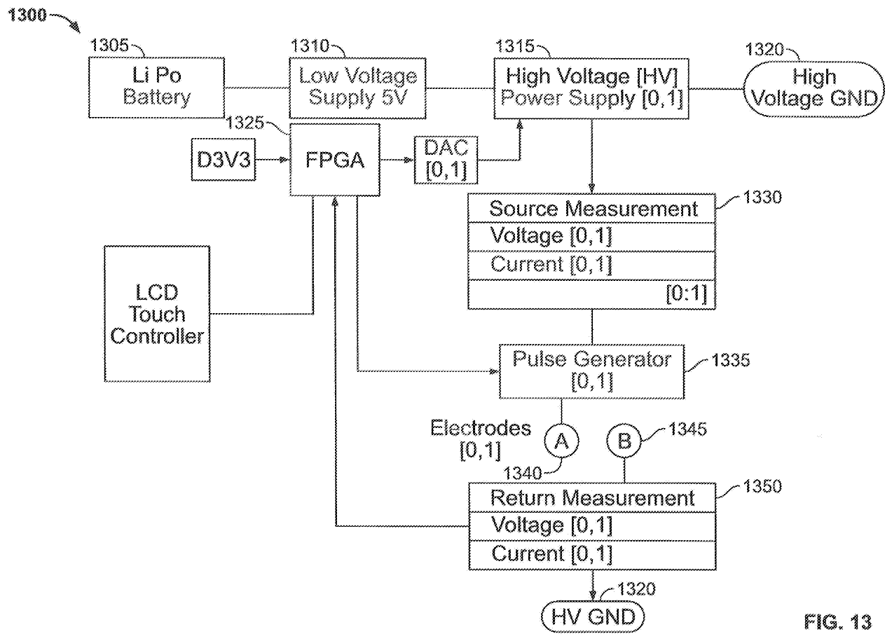

FIG. 13 is a block diagram of an embodiment of a circuit 1300 that can measure the dynamic properties of the electrodes in a channel, such as current, voltage, resistance, capacitance, and/or inductance according to some embodiments. In some embodiments, a battery 1305 connects to a low voltage power supply 1310 (e.g., 5 V, which supplies the 3.3 V identified above), which connects to a high voltage (HV) power supply 1315. In some embodiments, the HV power supply 1315 connects to ground 1320. In some embodiments, the HV power supply 1315 provides the sense pulse 1025. In one embodiment, the HV power supply 1315 also provides the stimulation pulse 1030. In some embodiments, a field-programmable gate array (FPGA) 1325 connects to a digital-to-analog converter (DAC), which connects to the HV power supply 1315. In some embodiments, the FPGA 1325 is a massively parallel microcontroller computer—a programmable analog chip with a program burned onto it. In some embodiments, The FPGA 1325 is based on a clock and is completely analog. Thus, in some embodiments, there is no time division or multiplexing. Although described as a FPGA, any programmable logic device (PLD) can be used according to some embodiments. In some embodiments, the FPGA 1325 also connects to a digital power supply D3V3.

In some embodiments, the HV power supply 1315 can obtain source measurements (e.g., voltage or current), as shown in block 1330. In some embodiments, source measurement block 1330 is a source measurement circuit. In some embodiments, a pulse generator 7335 connects to electrode A 1340 (the transmitting electrode/sensor in this instance). In some embodiments, the pulse generator 7335 is connected to the FPGA 7325.

In some embodiments, Electrode B 1345, the return electrode/sensor representing the output, connects to a return measurement block (or circuit) 1350, which also connects to the FPGA 7325 and HV ground 1320. In some embodiments, the return measurement block/circuit 1350 is identical to the source measurement block/circuit 1330. In some embodiments, the FPGA 7325 also connects to an LCD touch controller for controlling the circuit 1300.

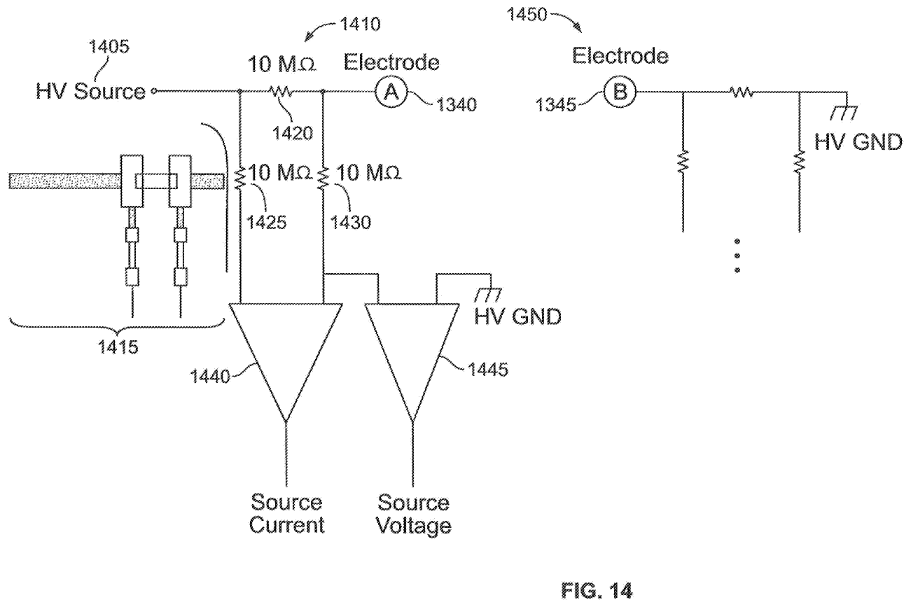

Referring to FIG. 14, the analog sense circuit to measure the source voltage and source current is shown according to some embodiments. In some embodiments, an HV source 1405 is applied to a resistor network 1410 connected to a shunt 1415. In some embodiments, a first resistor 1420 is a 10 W 0.1% resistor and is connected to a second and third resistor 1425, 1430 that are, in one embodiment, 1 MW 0.01% resistors. In some embodiments, the resistor network 1410 is connected to the electrode A 1340. In some embodiments, the shunt 1415 is connected to a wide trace in and wide trace out for power with a pull-up tap. In some embodiments, the resistors 1425, 1430 are connected to a first operational amplifier (op-amp) 1440 to measure source current. In some embodiments, resistor 1430 is connected to a second op-amp 1445 to measure source voltage. In some embodiments, the other side of the circuit (circuit 1450) is connected to electrode B 1345 and is the same circuit as the circuit with resistor network 1410, shunt 1415, and op-amps 1440 and 1445. Thus, in some embodiments, these circuits enable measurement of input power and output power. Although the resistors 1420, 1425, 1430 are shown with particular values, in some embodiments, these values are arbitrary and any corresponding resistor values can be used.

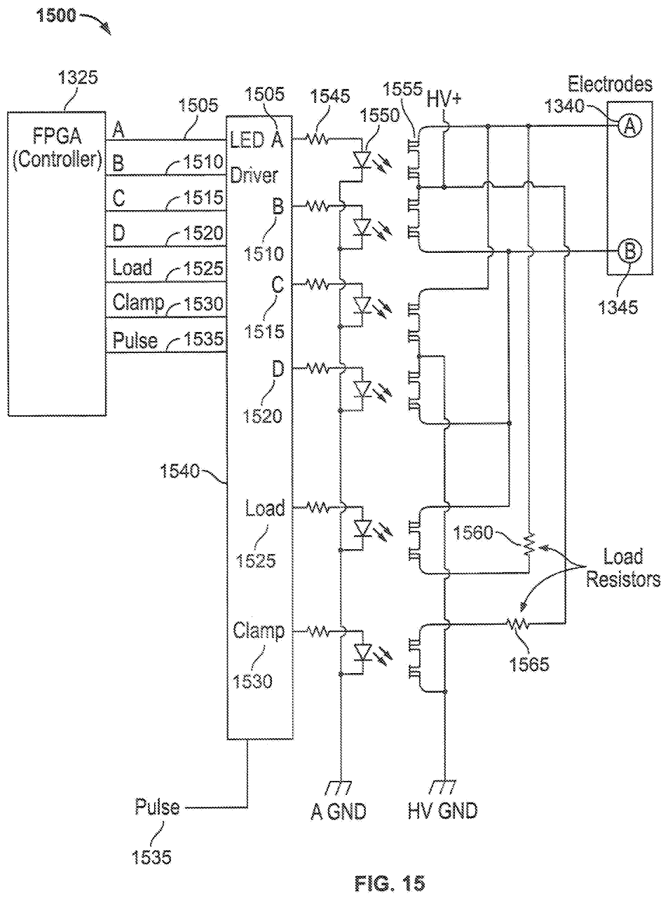

FIG. 15 is an embodiment of a circuit 1500 to generate a stimulation pulse 1030 according to some embodiments. In some embodiments, the circuit 1500 uses optically coupled FETs (also referred to as solid state relays (SSRs) or optoFETs) to generate the stimulation pulse 1030 because of a low electromagnetic interference (EMI) waveform generated by the circuit 1500. In some embodiments, this prevents interference with precision instruments and medical equipment so that this circuit (and, therefore, a brace utilizing this circuit) can be used in the operating room or near sensitive medical equipment.

As stated above, in some embodiments, the circuit 1500 includes a controller, which can be an FPGA 1325. In some embodiments, the controller 1325 includes an A output 1505, a B output 1510, a C output 1515, a D output 1520, a LOAD output 1525, a CLAMP output 1530, and a PULSE output 7535. In some embodiments, these outputs are optionally provided to an LED driver 1540. In some embodiments, each output of the LED driver is connected to an LED resistor (hereinafter LED resistor 1545) and an LED (hereinafter LED 1550). In some embodiments, the LED 1550 is optically coupled to the SSR (hereinafter SSR 7555). As shown in circuit 1500, different SSRs 7555 are connected to electrode(s) 1340, 1345 according to some embodiments. In some embodiments, the circuit 1500 also includes two load resistors 1560, 1565.

In some embodiments, the LEDs 7550 turn power on and off in the circuit 1500. In some embodiments, the LEDs 7550 and SSRs 7555 are in a shielded light proof box (or encased in an integrated circuit) to electrically isolate those components of the circuit 1500. In some embodiments, the SSRs 7555 work on a voltage differential, and there is no reference from gate voltage to source or drain. In some embodiments, one SSR chip includes two SSRs and the corresponding LEDs.

In some embodiments, clamp 1530 is to clamp the power supply, so that when the voltage from the power supply needs to drop quickly, the clamp activates. In some embodiments, the clamp has to be released in order to drive the circuit 1500. Thus, in some embodiments, when the clamp 1530 and load resistors 1560, 1565 are engaged, the load is applied across the electrodes 1340, 1345 and if the system experiences a failure or an out of range value, the circuit 1500 will fail safe and nothing harmful will happen to the patient. In some embodiments, this safety feature enables the brace to be worn at all times, without worrying about where the patient is located (e.g., driver or passenger of automobile, in a swimming pool, etc.). In some embodiments, the circuit 1500 will not just turn on or send a stimulation pulse without adequate and proper activation. In some embodiments, if there is a short circuit, the circuit 1500 applies a load across the electrodes 1340, 1345. In some embodiments, if the patient fell into a pool wearing a device utilizing circuit 1500, the device would fail safe. In other words, in some embodiments, the patient would not be harmed if this occurred (or if any out of range input was provided to one or both of the electrodes 1340, 1345). Thus, unlike conventional systems, which often require the user to increase the power being input to the muscles or human tissues after a certain amount of time, in some embodiments, this system recognizes an out of bounds signal and often results in a decrease in resistance (as you activate muscle, ion channel through muscle increases) and power due to power dissipation after the initial sense pulse. Thus, in some embodiments, the system minimizes pain experienced by the patient because of the closed loop nature of the system and the decision making process that occurs after each pulse.

When a signal is applied, the SSRs 7555 close and complete the circuit. In some embodiments, the load resistors are typically closed and only open when the system powers up. In some embodiments, the LED resistors 1545 are typically open. In some embodiments, the circuit 1500 doesn't allow a high voltage supply to come up to a high voltage because the load resistors 1560, 1565 are held across it and force the high voltage supply to shut down. In some embodiments, this removes many single points of failure. In some embodiments, the high voltage power supply can also sense overcurrent.

In some embodiments, when CLAMP 1530 is high (active), this removes the load resistor 7565 from the circuit. In some embodiments, when LOAD 7525 is high (active), it removes the load resistor 1560 from the circuit. Thus, in some embodiments, the LOAD 7525 applies load resistor 1560 across the electrodes 1340, 1345. In some embodiments, CLAMP 1530 clamps them to a high voltage power supply. In some embodiments, this setup can help with calibration. In some embodiments, the load resistors 1560, 1565 are lOW power resistors.

In some embodiments, the optional LED driver 1540 is a digital buffer that sources current Ito drive the LEDs 7550. PULSE 7535 is active low. In some embodiments, the LED driver 1540 polls the PULSE signal 7535 and then sets the direction bits. In some embodiments, to generate a stimulation pulse 1030, one way is to have A 7505 high (high voltage to electrode A 1340) and D 1520 high. In some embodiments, this will cause current to flow in one direction (e.g., from electrode A 1340 to electrode B 1345). In some embodiments, if C 7575 is high and B 1510 is high, current flows the other way (e.g., from electrode B 1345 to electrode A 1340).

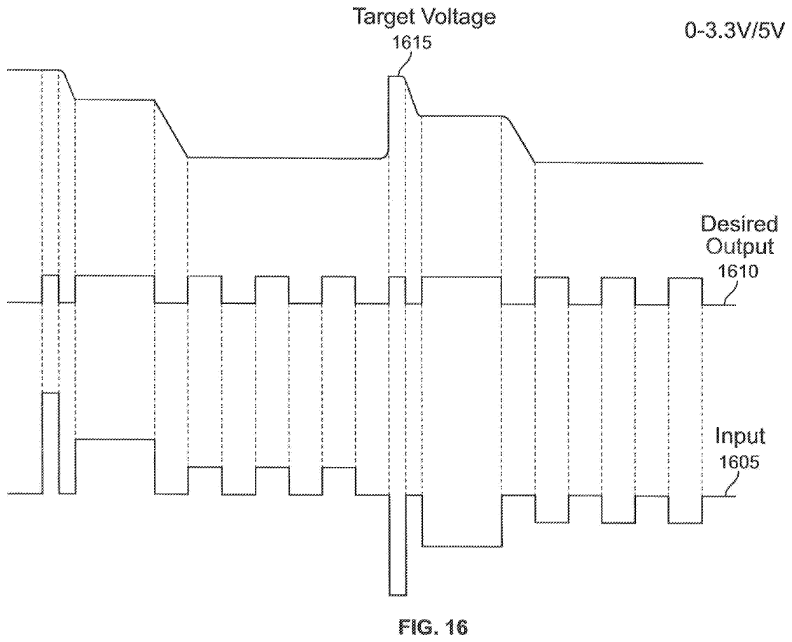

FIG. 16 is an embodiment of the input waveform 1605, a desired output waveform 1610, and a target voltage 7675 according to some embodiments. In some embodiments, the dashed lines are reference lines. In some embodiments, the input waveform 1605 is the same waveform as shown in FIG. 10A (with the sense pulse 1025 and the stimulation pulse 1030). In some embodiments, the desired output waveform 1610 includes a pulse for each sense and stimulation pulses 1025, 1030. The target voltage 7675 is a voltage for the 0-3.3. V/5 V reference voltage as identified above. This is different than a typical PWM signal in that it is a pure analog signal.

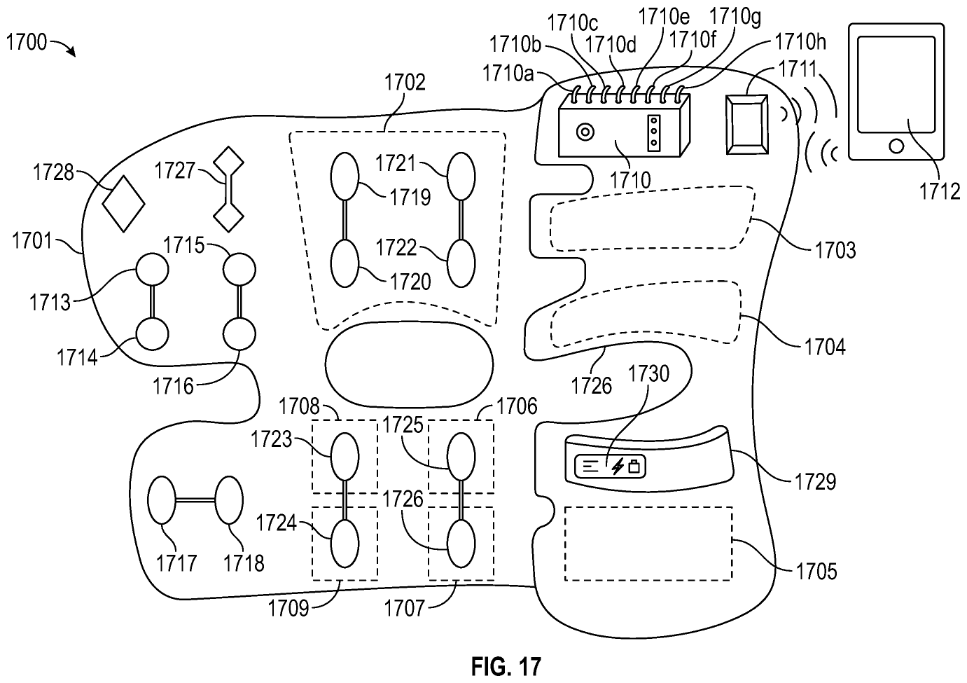

FIG. 17 illustrates an occlusion system 1700 with one or more fluid bladders 1702-1709 coupled to a knee brace 1701 according to some embodiments. In some embodiments, one or more fluid bladders 1702-1709 are configured to selectively apply pressure to one or more portions of a user's leg to restrict bloodflow therethrough. As shown in FIG. 17, the one or more fluid bladders 1702-1709 are each bladder is selectable and configurable using GUI 1712 to apply pressure by providing fluid from pump 1710 to a specific zone of the limb through fluid tubes 1710a-1710h according to some embodiments. In some embodiments, the occlusion system 1700 includes one or more continuous fluid bladders (not shown) that extend around the parameter of one or more portions of the occlusion system 1700 that is configured to apply blood flow restriction to the circumference of an entire limb. Although the garment shown in FIG. 17 is a closable knee brace wrap 1700, in some embodiments the garment is a sleeve garment similar to FIG. 9: 920, and is continuous around an outer perimeter of a central opening.

Advantageously, the one or more fluid bladders 1702-1709 are placed on top of one or more EMS system components such as senor pairs 1713, 1714; 1715, 1716; 1717, 1718; 1719, 1720; 1721, 1722; 1723, 1724; 1725, 1726. In some embodiments, the positioning one or more fluid bladders 1702-1709 over one or more sensor pairs 1713-1726 improves connectivity between one or more sensor pairs by forcing the one or more sensor pairs against the skin. For example, fluid bladder 1705 is configured to be positioned over sensor pair 1717, 1718; fluid bladder 1702 is positioned over sensor pairs 1719, 1720 and 1721, 1722; and/or fluid bladders 1706-1709 are each positioned over a respective sensor 1723-1726 according to some embodiments. In some embodiments, selectively applying pressure using one or more sensor pairs 1713-1726 mitigates the effects of a user not tightening the occlusion system 1700 using securing straps (e.g., Velcro® straps 1726) because of pain and/or discomfort according to some embodiments. In some embodiments, the system is configured to sense weak connectivity between one or more sensor pairs 1713-1726 and pressurize the one or more fluid bladders 1702-1709 with fluid (e.g., air) to apply pressure to the one or more sensor pairs 1713-1726 until a predetermined signal strength is obtained and/or a predetermined pressure is reached. In some embodiments, the system is configured to display an error message on GUI 1712 if connectivity does not improve.

Advantageously, the system is configured to relieve pressure around a wound by selectively pressurizing one or more bladders 1702-1709 (e.g., 1706, 1707, 1708) while leaving one or more other bladders 1702-1709 (e.g., 1709) unpressurized. In some embodiments, this allows for a more secure coupling of the brace to the skin while simultaneously not applying pressure to a portion of a wound located below the one or more other bladders 1702-1709.

In some embodiments, the system is configured to combine electrical muscle stimulation therapy and blood flow restriction in one device. In some embodiments, the electrical muscle stimulation therapy is configured to provide strengthening of the muscles using electrical stimulation while simultaneously measuring the effectiveness of the muscle stimulation using one or more sensor pairs 1713-1726. In some embodiments, the system is configured to enhance muscle adaptation and growth by at least partially occluding the blood flow through the limb while simultaneously applying sense and stimulation pulses 1025, 1030. In some embodiments, the amount of pressure applied by the limb is specific to the patient and/or section of a user's body to which the occlusion is being applied. In some embodiments, the amount of pressure applied by the one or more fluid bladders is between 30% and 90% of a patient and/or limb's limb occlusion pressure (LOP), which is the amount of pressure required to stop the flow of blood past the occlusion system 1700. In some embodiments, 30% to 70% LOP applied in conjunction with muscle stimulation applied by one or more sensor pairs 1713-1726 configured to cause a muscle contraction equivalent to 20% to 50% of a patient's single repetition maximum (1RM) lift is sufficient to enhance muscle adaptation and growth.

In some embodiments, the occlusion system includes one or more monitor systems 1711 configured to monitor the blood flow through the limb where the occlusion system is attached and/or transmit data to computer 300 and/or GUI 1712. In some embodiments, the system includes one or more conventional electromyography (EMG) electrodes 1727 configured to detect muscle movement and/or intensity. In some embodiments, the system includes one or more conventional force gauge sensors 1728 configured to measure muscle contraction. In some embodiments, one or more EMG electrodes 1727 and/or force gauge sensors 1728 are placed adjacent to one or more sensor pairs 1713-1726 to measure the effectiveness of the electrical stimulation.

In some embodiments, the occlusion system 1700 starts a process of determining pressure with one or more fluid bladders 1702-1709 in a deflated configuration. In some embodiments, electrostimulation therapy is applied, and the muscle contraction and/or response is recorded by the computer 300 and/or displayed on the GUI 1712. In some embodiments, the occlusion system 1700 at least partially fills the one or more fluid bladders 1702-1709 and applies another sense and stimulation pulses by using one or more sensor pairs 1713-1726. In some embodiments, the muscle contraction and/or response is detected by one or more force gauge sensors 1727 and/or EMG electrodes 1728 and recorded by the computer 300 and or displayed on the GUI 1712. In some embodiments, as the one or more fluid bladders 1702-1709 pressure increases, the occlusion system 1700 is configured to detect muscle contraction and/or response (e.g., mean motor unit spike and/or frequency) increase as blood flow decreases. In some embodiments, the system is configured to stop filling the one or more fluid bladders 1702-1709 when the muscle contraction and/or response stops increasing. In some embodiments, the system is configured to stop filling the one or more fluid bladders 1702-1709 at a pre-determined setpoint. In some embodiments, the GUI 1712 is configured to display one or more controls to allow a user to stop the fluid bladders manually.

In some embodiments, the monitoring system 1711 includes one or more ultrasound systems 1729. In some embodiments, the ultrasound system 1729 is positioned adjacent to one or more sensor pairs 1713-1726 (e.g., sensor pair 1717-1718) and/or one or more fluid bladders 1702-1709 (e.g., fluid bladder 1705). In some embodiments, the ultrasound system 1729 is in communication with computer 300. In some embodiments, at least a portion of the ultrasound system 1729 is positioned within one or more occlusion system 1700 pockets and/or is embedded in the fabric. In some embodiments, the ultrasound system 1729 comprises one or more conventional ultrasound devices. In some embodiments, the ultrasound system 1729 is configured to monitor the flow of blood through a user's limb. In some embodiments, the ultrasound system 1729 is configured to display blood flow on one or more displays 1730 and/or GUIs 1712. In some embodiments, the occlusion system 1700 is configured to control one or more fluid bladders 1702-1709 and/or one or more sensor pairs 1713-1726 based on the blood flow through a user's limb. In some embodiments, the system is configured to adjust one or more fluid bladders 1702-1709 to achieve a predetermined blood flow based on one or more ultrasound system 1729 measurements. In some embodiments, the ultrasound system 1729 comprises one or more conventional probe surfaces (not shown) in direct contact with at least a portion of the user's skin.

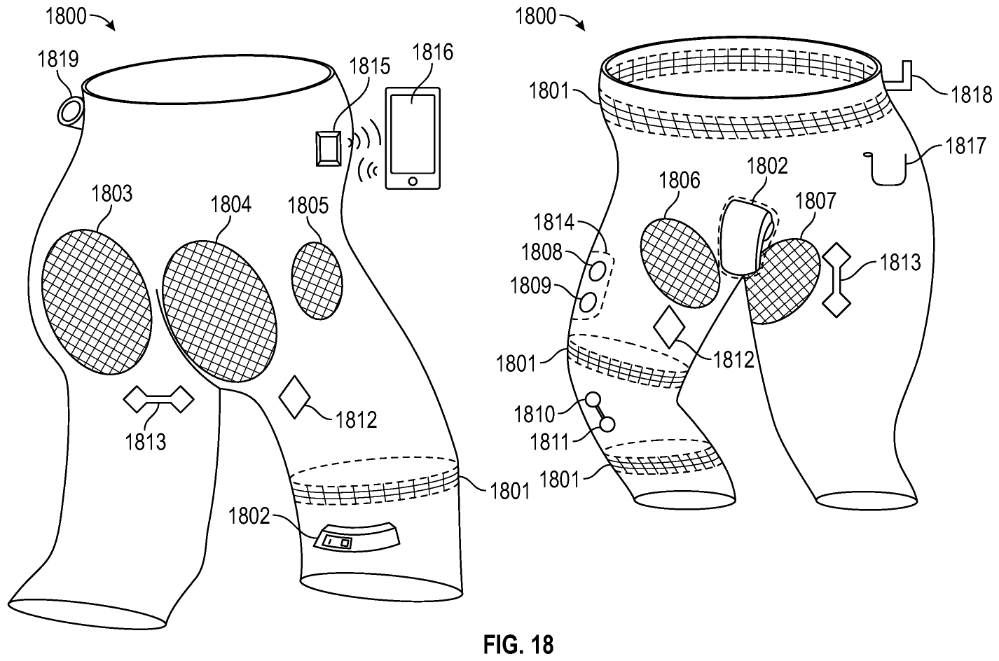

FIG. 18 illustrates a pelvic stimulation portion of the system 1800 according to some embodiments. In some embodiments, the pelvic stimulation system 1800 includes one or more occlusion systems 1801, ultrasound systems 1802, the electrostimulation systems 1803-1807, monitor systems 1808, GUIs 1809, sensor pairs 1808-1811, force gauge sensors 1812, EMG sensors 1813, and/or computers 300. The operation of the components are similar to respective components previously described and will not be repeated in the interest of being concise.

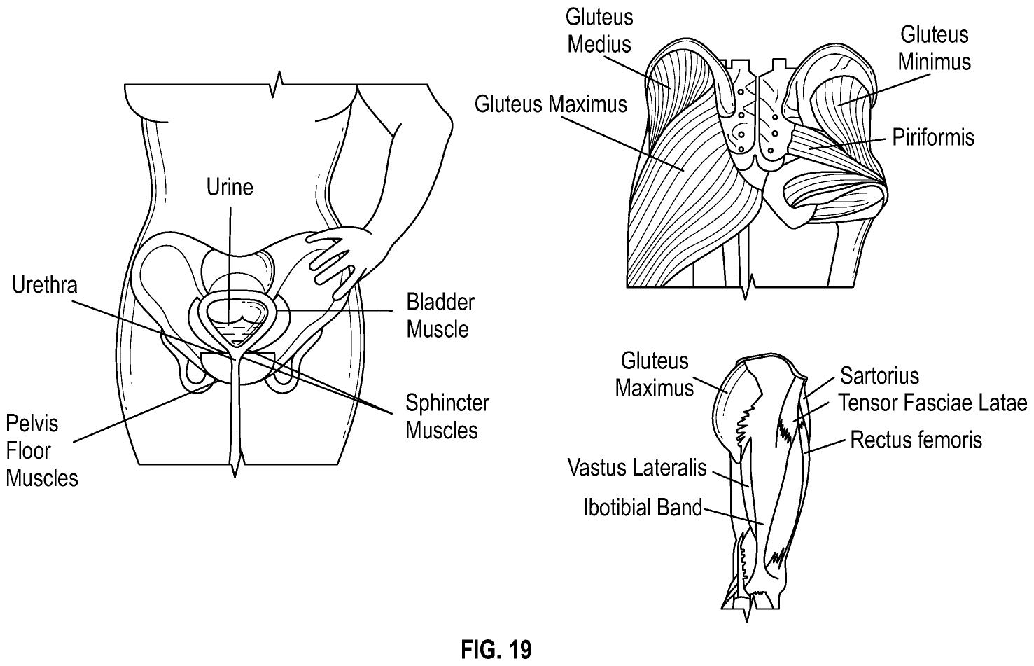

In some embodiments, the pelvic stimulation system 1800 is configured to stimulate the pelvic floor muscle (see FIG. 19) to create a contraction of one or more pelvic floor muscles to re-educate and/or treat weakness or disuse atrophy associated with stress urinary incontinence syndrome. In some embodiments, the pelvic stimulation system 1800 is configured to improve partially de-nervated urethral and pelvic floor musculature by enhancing the process of re-innervation via pelvic floor muscle stimulation. In some embodiments, the pelvic stimulation system 1800 is configured to apply neuromuscular electrical stimulation using, for example, one or more of electrodes 1803-1807 to contract the pelvic floor muscles. In some embodiments, pelvic stimulation system 1800 is configured to at least partially measure pelvic floor muscle contraction using one or more EMG sensors 1813. In some embodiments, pelvic stimulation system 1800 is configured to send an image of one or more anatomical structures shown in FIG. 19 to GUI 1809 and/or computer 300, and/or a patient mobile app. In some embodiments, the patient mobile app is configured to enable one or more of neuromuscular electrical stimulation management, low back pain related clinical surveys, neuromuscular electrical stimulation compliance, machine learning methods for personalized neuromuscular electrical stimulation dose, and a provider portal. In some embodiments, the neuromuscular electrical stimulation applied by one or more electrodes 1803-1805 is configured to improve gluteus muscle strength. In some embodiments, the occlusion system 1801 is configured to limit the flow of blood to one or more gluteus muscles.

In some embodiments, the pelvic stimulation system 1800 is configured to be applied non-invasively from outside the pelvis region using cutaneous surface electrodes 1803-1807. In some embodiments, the cutaneous surface electrodes 1803-1807 include all the functionality of any sensor pair described herein. In some embodiments, addition sensor pairs 1808-1811 are positioned at predetermined locations, and can include one or more fluid bladders 1814 configured to apply pressure to the sensor pairs and/or cutaneous surface electrodes 1803-1807.

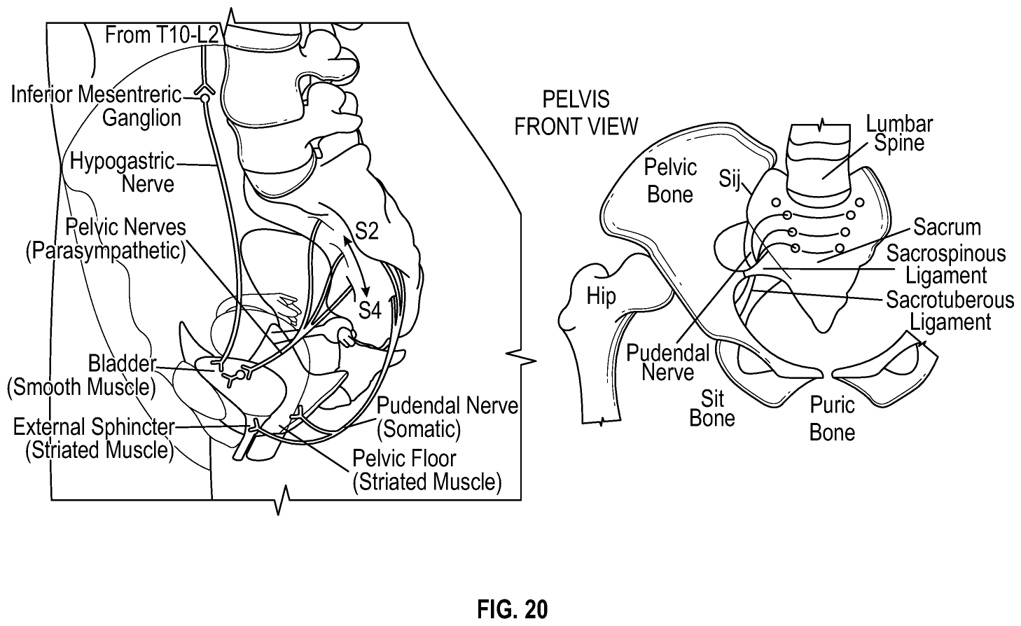

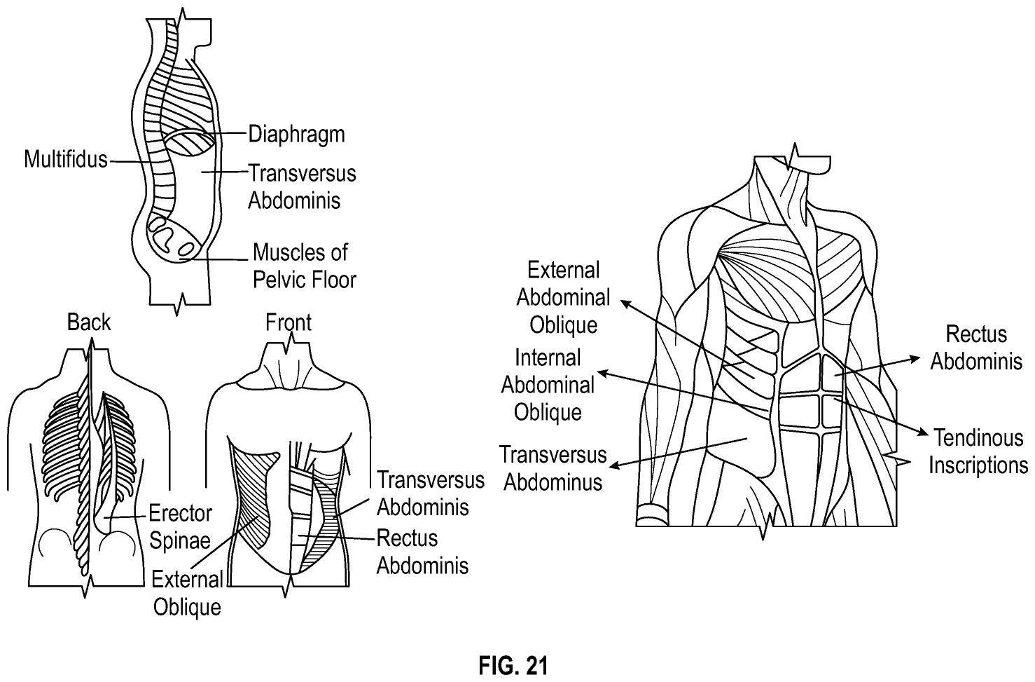

In some embodiments, one or more electrodes 1803-1807 placed externally over the pelvis region are configured to stimulate the pudendal nerve (see FIG. 20) and at least partially cause urethral closure by activating the pelvic-floor muscles (see FIG. 19). In some embodiments, the cutaneous surface electrodes 1803-1807 are configured to stimulate the motor neurons of the pudendal nerve and pelvic floor muscles (See FIG. 20). In some embodiments, the pelvic floor stimulates the gluteus muscles including one or more of the gluteus maximum and/or gluteus medius. In some embodiments, the system is configured to create contractions of the gluteus muscles to treat, re-educate, and/or rebuild the gluteus muscles over a period of time. In some embodiments, the system is configured to treat atrophy associated with hip osteoarthritis, hip surgical procedures, and/or any other clinical condition associated with gluteus weakness.

In some embodiments, the system comprises electrode flaps 1817 and/or hooks 1818 and/or loop fasteners 1819 configured to secure one or more electrodes, monitor systems, and/or sensors and/or are configured to enable replacement of one or more electrodes and/or sensors assembly components. In some embodiments, the system is configured to provide neuromuscular electrical stimulation waveform, closed-loop feedback, and power-controlled output using one or more electrodes and/or sensors, as illustrated in FIGS. 12 and 16.

Although the garment in FIG. 18 is shown as shorts, In some embodiments, the pelvic stimulation system 1800 (i.e., one or more sensor pairs) is provided in any type of garment (e.g., wraps, sleeves, cuffs, and/or individual patches). In some embodiments, pelvic stimulation system 1800 includes two to six pre-positioned electrodes configured and arranged to stimulate one or more of the anterior, posterior, and/or lateral regions of the pelvis. In some embodiments, the pelvic stimulation includes one or more electrodes (e.g., electrodes 1803-1807) that cover a combined region between 60% and 100% of the total amount of user's skin that covers the surface of the gluteal muscles. In some embodiments, system comprises two electrodes positioned on the right gluteal maximus, two electrodes positioned on the left gluteal maximus, one electrode positioned on the right gluteal medius, and/or one electrode positioned on the left gluteal medius (see FIG. 19). In some embodiments, one or more sensors are configured to provide real-time stimulation biofeedback to computer 300 by measuring the intensity of the muscle contractions (as described above) to measure the effectiveness of the muscle contraction.

In some embodiments, different treatment factors such as waveform shape, stimulus frequency, intensity, pulse duration, number of treatments per day, and treatment session length impact the treatment outcomes. In some embodiments, to activate the motor neurons, the stimulation frequency is applied in 20-50 Hz range using one or more electrodes and/or sensor pairs to contract one or more pelvic muscles and/or stimulate one or more nerves in the pelvic region. In some embodiments, the stimulation intensity is managed by the patient via the computer 300 and/or GUI 1816. In some embodiments, the pelvic stimulation system 1800 is configured to apply an output voltage in a range from 0 to 20 V_{RMS}. In some embodiments, the pelvic stimulation system is configured to apply a monophasic waveform. In some embodiments, the pelvic stimulation system 1800 is configured to apply a pulse duration in a range from 0 to 5 milliseconds. In some embodiments, the system is configured to apply a pulse duration based on the amount of muscle contraction detected by the monitoring system (e.g., one or more of the EMG sensors, force gauge sensors, and/or ultrasonic monitoring systems) as described herein.

In some embodiments, the computer 300 is configured to analyze one or more images received from the ultrasonic monitoring system 1802 and increase and/or decrease the electrostimulation to the glute region (e.g., electrodes 1803-1805) based on the pelvic muscle response. In some embodiments, the system includes artificial intelligence (AI) and/or machine learning to analyze the one or more images received from one or more ultrasonic monitoring systems described herein. In some embodiments, the AI and/or machine learning is configured to determine if a given applied electric stimulation has stopped the flow of urine from leaving the bladder.

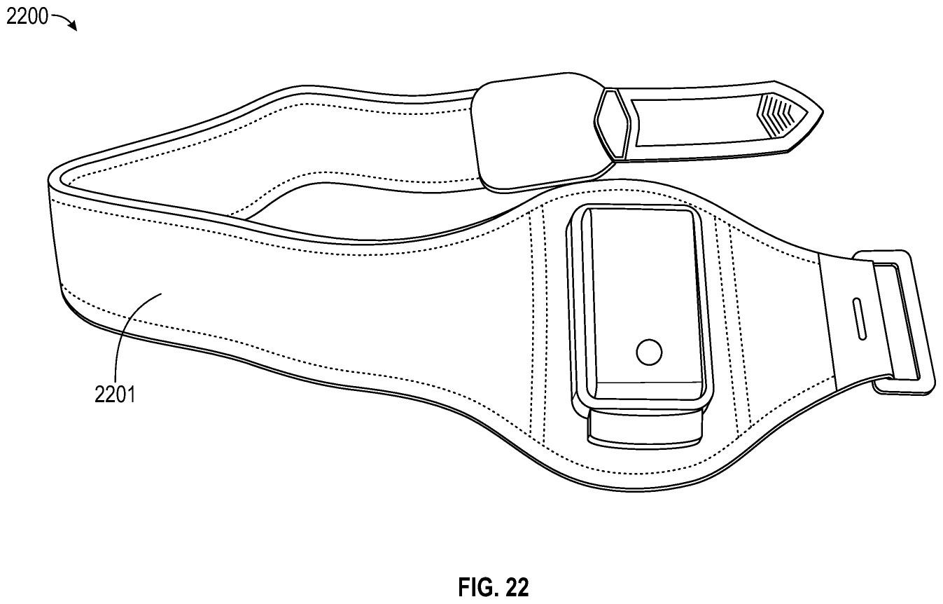

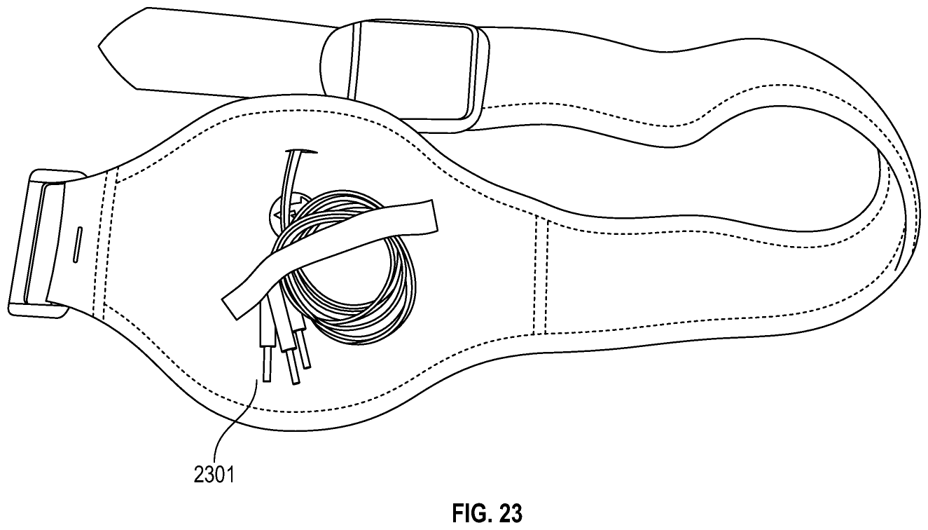

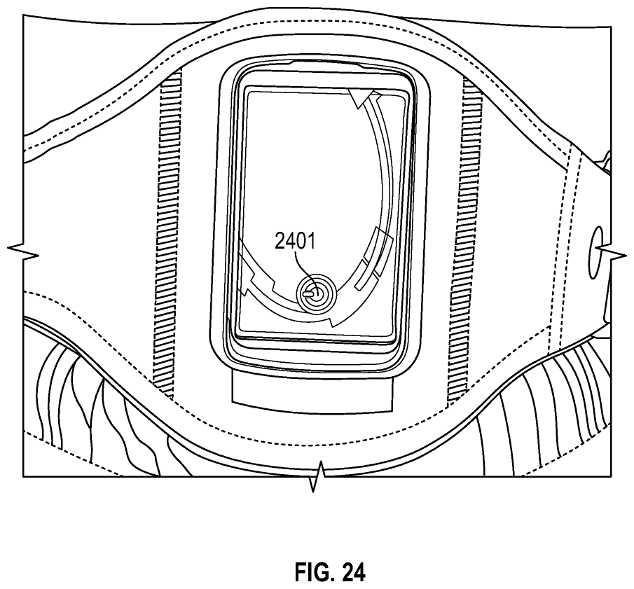

FIGS. 22-24 show the occlusion system of FIG. 17 as a wrap 2200 (i.e., band, belt) according to some embodiments. In some embodiments, one or more occlusion system components are arranged to control and/or monitor the flow of blood and/or stimulate and monitor specific muscles as needed. In some embodiments, the wrap 2200 includes a fluid bladder 2201 that extends along the entire length of the wrap 2200. In some embodiments, the wrap is configured to reduce blood flow to one or more portions of a user's anatomy. In some embodiments, the wrap 2200 includes one or more electrical leads to connect one or more electrodes, sensors, and/or computers described herein. In some embodiments, the wrap includes one or more controllers 2401 to control one or more occlusion system components.

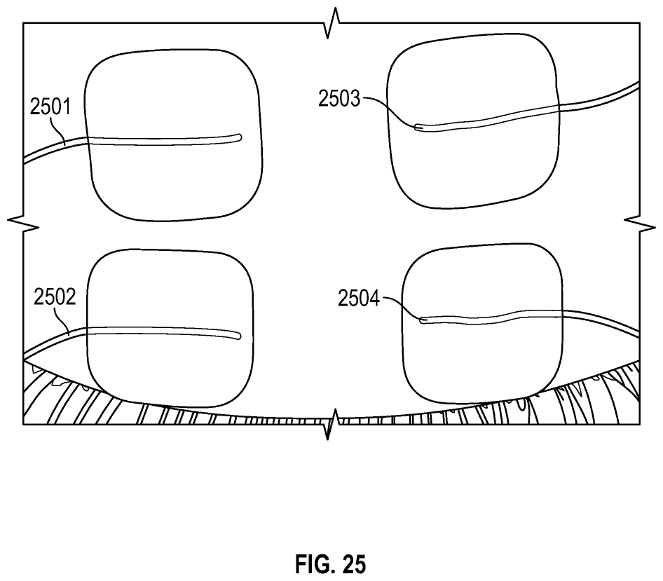

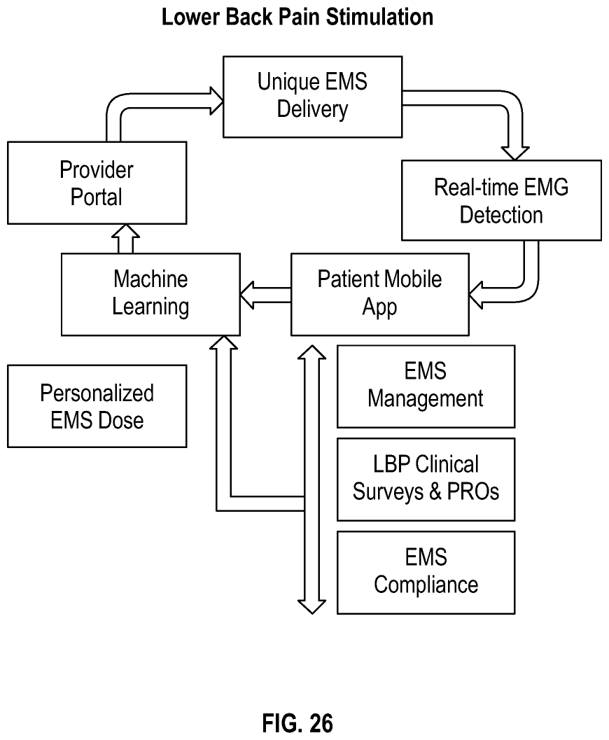

FIG. 25 shows sensor pairs 2501-2504 configured and arranged on a user's lower back above the waistline for lower back pain treatment with LAMES according to some embodiments.

FIG. 26 is a flowchart illustrating one or more steps executed by the mobile app according to some embodiments.

The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Some embodiments of the system are presented with specific values and/or setpoints. These values and setpoints are not intended to be limiting, and are merely examples of a higher configuration versus a lower configuration and are intended as an aid for those of ordinary skill to make and use the system. In addition, “substantially” and “approximately” when used in conjunction with a value encompass a difference of 10% or less of the same unit and scale of that being measured. In some embodiments, “substantially” and “approximately” are defined as presented in the specification.

“Simultaneously” as used herein includes lag and or latency times associated with a conventional computer attempting to process multiple types of data at the same time.

Acting as Applicant's own lexicographer, Applicant defines the use of and/or, in terms of “A and/or B,” to mean one option could be “A and B” and another option could be “A or B.” Such an interpretation is consistent with ex parte Gross, where the USPTO Patent Trail and Appeal Board established that “and/or” means element A alone, element B alone, or elements A and B together.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Modifications to the illustrated embodiments and the generic principles herein can be applied to all embodiments and applications without departing from embodiments of the system. Also, it is understood that features from different embodiments presented herein can be combined to form new embodiments that fall within the disclosure. Thus, embodiments of the system are not intended to be limited to embodiments shown but are to be accorded the widest scope consistent with the principles and features disclosed herein. The figures, which are not necessarily to scale, depict some embodiments and are not intended to limit the scope of embodiments of the system.

We claim:

1. A garment system comprising:
an occlusion system comprising one or more fluid bladders, and
an electrostimulation system comprising one or more electrodes;
wherein the occlusion system is configured to reduce the flow of blood to at least a portion of a user's body while the electrostimulation system simultaneously applies electrical stimulation to one or more muscles within an area of the reduced the flow of blood.

2. The garment system of claim 1,
wherein the at least one fluid bladder is placed on top of at least one of the plurality of electrodes.

3. The garment system of claim 1,
wherein the at least one fluid bladder is placed adjacent to at least one of the plurality of electrodes.

4. The garment system of claim 1,
wherein the electrostimulation system comprises at least one sensor comprising a plurality of electrodes including at least one active electrode and at least one receiving electrode, the at least one sensor configured and arranged to be in physical contact with skin of a patient forming an electrical circuit with control electronics of at least one controller, the electrical circuit configured and arranged to measure an electrical parameter using the at least one active electrode and at least one receiving electrode, and to form a closed loop electrical muscle stimulation system;

5. The garment system of claim 4,
wherein a stimulation current or voltage applied by the sensor onto the skin between the at least one active electrode and at least one receiving electrode is based on at least one program and at least one electrical parameter measured through the at least one active electrode and at least one receiving electrode.

6. The garment system of claim 5,
wherein the at least one controller is configured to:
(a) apply a sense electrical pulse to the tissue using the at least one sensor,
(b) measure the at least one electrical parameter from the tissue,
(c) using at least one of the active electrodes, adjustably apply a stimulation pulse to the tissue based at least in part on the measured electrical parameter, the stimulation being adjustably controlled by the at least one controller to maintain a constant power output to the tissue based at least in part on the at least one electrical parameter, and
(d) repeat steps (a)-(c).

7. A garment system comprising:
a pelvic stimulation system comprising one or more electrodes;
wherein the pelvic stimulation system is coupled to one or more garments;
wherein the pelvic stimulation system is configured to reduce the flow of urine from a user's bladder by contracting the user's pelvic floor muscles using electrical stimulation produced by the one or more electrodes.

8. The garment system of claim 7,
wherein the electrodes are configured to apply the electrical stimulation from outside a user's body.

9. The garment system of claim 7,
wherein the electrodes are configured to apply the electrical stimulation to the surface of a user's skin.

10. The garment system of claim 7,
further comprising one or more electromyography (EMG) sensors;
wherein the one or more EMG sensors are arranged adjacent at least one of the one or more electrodes.

11. The garment system of claim 10,
wherein the one or more EMG sensors are configured and arranged to detect and/or monitor muscle movement and stimulation intensity in a user's pelvis.

12. The garment system of claim 7,
further comprising one or more force gauge sensors;
wherein the one or more force gauge sensors are arranged adjacent at least one of the one or more electrodes.

13. The garment system of claim 12,
wherein the one or more force gauge sensors are configured and arranged to detect and/or monitor muscle movement and stimulation intensity in a user's pelvis.

14. A garment system comprising:
an occlusion system,
a pelvic stimulation system, and
a monitoring system;
wherein the occlusion system, the pelvic stimulation system, and the monitoring system are each coupled to a garment; and
wherein the garment configured to fix and/or secure to at least a portion of a user's body.

15. The garment system of claim 12,
wherein the pelvic stimulation system is configured to reduce the flow of urine from a user's bladder by contracting the user's pelvic floor muscles using electrical stimulation produced by the one or more electrodes.

16. The garment system of claim 12,
wherein the occlusion system is configured to reduce the flow of blood to at least a portion of a user's body while the electrostimulation system simultaneously applies electrical stimulation to one or more muscles with an area of the reduced the flow of blood.

17. The garment system of claim 12,
wherein the monitoring system comprises an ultrasound monitoring system.

18. The garment system of claim 17,
wherein the ultrasound monitoring system is configured to measure the flow of blood through an area of restricted blood flow;
wherein the occlusion system is configured to cause the restricted blood flow.

19. The garment system of claim 17,
wherein the ultrasound monitoring system is configured to measure the contraction of pelvic floor muscles.

20. The garment system of claim 17,
wherein the ultrasound monitoring system is configured to measure urine flow from a user's bladder.
